(19) United States
(12) Patent Application Publication
(10) Pub. No.: US 2008/0293625 A1
(43) Pub. Date: Nov. 27, 2008

(54) Diagnosis of Inflammatory Bowel Diseases, More Particularly Ulcerative Colitis

(75) Inventors: Winfried Stocker, Gross Gronau (DE); Swantje Mindorf, Burg (DE); Lars Komorowski, Ratzeburg (DE)

Correspondence Address: Ballard Spahr Andrews & Ingersoll, LLP, SUITE 1000, 999 PEACHTREE STREET, ATLANTA, GA, 30309-3915, US

(73) Assignee: EUROIMMUN Medizinische Labordiagnostika AG, Lubeck (DE)

(21) Appl. No.: 10/599,942

(22) PCT Filed: Apr. 15, 2005
(86) PCT No.: PCT/EP05/03978

§ 371 Date: Jul. 30, 2007

(30) Foreign Application Priority Data
Apr. 15, 2004 (DE) 10 2004 018 418.6

(51) Int. Cl. A61K 38/00; C07K 14/00; C07K 16/18; A61P 43/00; G01N 33/53

(52) U.S. Cl. 514/12; 530/350; 530/388.1; 435/7.21

(57) ABSTRACT

The present invention concerns goblet cell antigen, a method for detecting antibodies directed against goblet cell antigen, a method based thereon for diagnosis of inflammatory bowel diseases and a kit for diagnosis of inflammatory bowel diseases, as well as monoclonal antibodies directed against goblet cell antigen.

The present invention relates to goblet cell antigen, a method for detection of antibodies against goblet cell antigen which is suitable for diagnosis of inflammatory bowel diseases, in particular colitis ulcerosa, and a kit for diagnosis of inflammatory bowel diseases, as well as monoclonal antibodies against goblet cell antigen.

The significance of inflammatory bowel diseases has increased in recent years due to an increasing incidence above all in western industrialized countries (Jenss et al., 1996, Morbus Crohn und Colitis ulcerosa. Informationen und Ratschläge, Serie Gesundheit [Crohn's disease and colitis ulcerosa. Information and advice, Health series] Piper/C & H.).

Colitis ulcerosa and Crohn's disease are chronic inflammatory intestinal diseases (CIID) which are characterized by inflammations which can affect various portions of the gastrointestinal tract to different degrees. In the case of colitis ulcerosa, in contrast to Crohn's disease, the inflammation reaction is limited to the large intestine and is therefore an important differentiating feature.

Both diseases are distinguished by an intermittent course, in which active phases of the disease with sometimes severe symptoms alternate with virtually symptom-free periods of time, which can last months or even years. Colitis ulcerosa and Crohn's disease are characterized by similar clinical symptoms. Nevertheless, the therapy and the medicaments necessary for alleviation differ for the two diseases. Differential diagnosis therefore acquires a particular importance.

Serological analysis gives first indications of the existence and severity of an inflammation. In this context, as a rule an increase in the typical inflammation parameters, such as blood sedimentation rate, C-reactive protein and leukocyte count, is detected.

The diagnosis of an inflammatory bowel disease can be made relatively quickly by the clinical symptoms (diarrhea and abdominal pain). The demarcation of CIID such as colitis ulcerosa and Crohn's disease from other diseases with similar clinical symptoms is important here. An infection with typical diarrhea pathogens (pathogenic Escherichia coli, Salmonellae etc.) can be ruled out by a bacteriological analysis of the stool.

Celiac disease or sprue is also a chronic diarrhea disease. However, this is caused by an intolerance of the small intestine towards the cereal protein gluten in combination with the enzyme transglutaminase and is characterized by atrophy of the mucosa of the small intestine. Since celiac disease sometimes also causes symptoms of an inflammatory bowel disease but cannot be treated with medicaments, an accurate diagnosis is very important. In cases of celiac disease, gluten acts together with transglutaminase as an allergen which triggers an antigen-antibody reaction which leads to destruction of the mucous membrane of the small intestine. With the aid of a biopsy from the small intestine and a serological analysis for autoantibodies against endomysium and gliadin by means of indirect immunofluorescence, a reliable demarcation is possible.

Sonography of the abdominal cavity is a relatively simple diagnostic measure. Thickenings of the intestinal wall and complications such as abscesses and fistulae (above all with Crohn's disease) can be detected by this method. However, sonography contributes to the diagnosis to only a small extent. Radiological investigation with contrast media is much more conclusive, but has been pushed into the background due to exposure to radiation. Further imaging methods which can be employed are computerized tomography (CT) and magnetic resonance tomography (MRT), which likewise make it possible to show changes in the region of the intestine.

The method of choice for differential diagnosis of colitis ulcerosa or Crohn's disease is still coloscopy, including a biopsy. In cases of colitis ulcerosa, a superficial inflammation is present, which is characterized by a uniform spread and an aboral increase and the formation of crypt abscesses. For Crohn's disease, a transmural inflammation reaction with which segmental, sharp-edged foci and granulomas occur is typical. An unambiguous diagnosis can therefore be made in most cases via the biopsy. Since patients with colitis ulcerosa are at increased risk of cancer of the large intestine, an annual examination of the large intestine by means of coloscopy is advised, in order to be able to resort to suitable therapeutic measures as quickly as possible if a carcinoma forms.

A further branch of diagnostics which is only rarely utilized by gastroenterologists is serological analysis of autoantibodies with the aid of an indirect immunofluorescence test (IIFT). Here, use is made of the fact that patients with Crohn's disease or colitis ulcerosa develop specific antibodies.

In the case of Crohn's disease, antibodies against the exocrine pancreas can be detected with a prevalence of 39% with IIFT; these do not occur in patients with colitis ulcerosa. Titers above 1:10 are pathognomic for Crohn's disease. Pancreas tissue from suitable primates serves as the substrate in this context, and in the positive case a “net-like granular, sometimes also drop-like fluorescence” is to be detected (Stöcker et al., 1987, Scand J Gastroenterol, 22: 41-52).

In one study, 76% of patients with colitis ulcerosa and 7% of patients with Crohn's disease had antibodies against an unknown antigen of the granulocytes. Swabs with human ethanol-fixed granulocytes show, with a positive result in the indirect immunofluorescence, a “smooth, sometimes also finely granular, perinuclear fluorescence of the cytoplasm (pANCA)” (Stöcker et al., 1987, ibid).

In 70% of patients with Crohn's disease and 8% of specimens from healthy blood donors, antibodies against Saccharomyces cerevisiae can be found in indirect immunofluorescence. The occurrence of these antibodies is independent of the development of the anti-pancreas antibodies. 80% of Crohn's disease patients can be detected by a combination of the two tests.

In 1959, autoantibodies against intestinal goblet cells were discovered in sera of patients with colitis ulcerosa (Broberger et al., 1959, J Exp Med, 110: 657-674), which could be responsible for the characteristic decrease in these cells in the course of the disease (Hayashi et al., 2001, Digestion 63: 28-31).

In one study, it was possible to specifically detect anti-goblet cell antibodies in 28% of patients with colitis ulcerosa (Stöcker et al., 1987, ibid). The significance of these autoantibodies for the pathogenesis is still unclear, as is the structure of the antigen. A clarification of the antigen structure could not only provide insights into the mechanism of the disease, it could also be the basis for the development of targeted therapy possibilities.

Human fetal intestinal tissue is ideal as a substrate for diagnostic investigation for autoantibodies against intestinal goblet cells by means of IIFT. Since the use is disputed from the ethical point of view and rat tissue leads to misleading results (Stöcker et al., 1984, Deutsche Medizinische Wochenschrift, 51/52: 1963-1969), however, as a rule intestinal tissue from suitable primates is employed in practice. In the positive case, a cloud-like, blurred fluorescence can be seen in a plane above the goblet cells (Stöcker et al., 1987, ibid). In addition to ethical considerations and the scarce availability of such substrates, there are also difficulties with reliability, as a result of which a very expensive and comprehensive quality control becomes necessary, however, a 100% reproducible quality cannot be ensured by these means, since it is a biological tissue which cannot be influenced. Frequent non-specific reactions render evaluation of the results difficult, so that these diagnostics can be carried out by only a few experts.

On the basis of the difficulties described, differing results have been achieved and published in the past. Investigation of these antibodies is therefore scarcely used for diagnosis of colitis ulcerosa, although the predictive value of a positive result is virtually 100%, and a colitis ulcerosa can be unambiguously detected with a positive test for anti-goblet cell antibodies. By a combination of the evidence from anti-goblet cell antibodies and pANCA, in 83% of the patients affected, colitis ulcerosa could actually be diagnosed serologically.

The development of a reliable test system, which is easy to evaluate and is based on a more easily accessible and more readily standardizable source of the target structure for anti-goblet cell antibodies, is therefore necessary in order to render a diagnosis and subsequent specific therapy of colitis ulcerosa based on investigation of these antibodies possible.

This problem is solved by the subject matter of claims 1 to 34, and in particular by the use of a goblet cell antigen which is obtainable by culture of HT29-18N2 cells under certain culture conditions and which reacts specifically with anti-goblet cell antibodies from colitis ulcerosa patients.

Attempts have already been made earlier to develop cell culture systems which allow a specific detection of antibodies in cases of colitis ulcerosa or Crohn's disease. Lee et al. (1999, Gut 44: 196-202) investigated the three colon carcinoma cell lines Caco-2, HT29 and LS-180 for their suitability as a source of the target structure for anti-goblet cell antibodies for a specific test system for detection of such antibodies. However, the results obtained with this system were not specific for colitis ulcerosa patients, but the cell lines investigated also reacted with antibodies from Crohn's disease patients.

Furthermore, it was shown for the cell line HT29 that, by exchange of glucose for galactose in the culture medium, it apparently irreversibly differentiates into various intestinal epithelial cells (Huet et al., 1987, JCB 105: 345-357). It was shown for a subclone, HT29-18N2, that in addition to the morphology of goblet cells, it also has some of their other properties, as, e.g., the capability to be stimulated by carbachol, which induces secretion of mucus. However, in other properties, the cell line HT29-18N2 is said to differ from goblet cells, for example in its growth properties or in the formation of intraepithelial lumina (Phillips et al., 1988, Gastroenterology 94: 1390-1403).

Hibi et al. (1994, Gut 35: 224-230) attempted to employ the cell line HT29-18N2 for diagnosis of inflammatory bowel diseases, but did not arrive at specific results, since sera both of patients with colitis ulcerosa (29-38%, depending on the method) and of those with Crohn's disease (33%) reacted with the cells used. The antigen recognized by the antibodies is said to have a molecular weight of >200 kDa, to comprise an individual polypeptide chain and to be recognized by the antibodies both in the native and in the reduced form. Further identification of this antigen was not undertaken.

It has furthermore been found that during culture in glucose-containing (3.0 g/l), protein-free medium (e.g. Protein Free Hybridoma Medium II, PFHM II, Invitrogen), the cell line HT29-18N2 predominantly grows as goblet cells filled with secretory granula, which, in contrast to the cell line HT29, form individual layers (Phillips et al., 1995, In Vitro Cell Dev Biol 31: 421-423). These cells moreover form a number of glycoproteins, so-called mucins, which are in some cases secreted.

Surprisingly, it has now been found that during culture of HT29-18N2 cells in the absence of protein, an antigen which reacts with anti-goblet cell antibodies from colitis ulcerosa patients is expressed. This antigen is called goblet cell antigen in the following.

The present invention for the first time renders targeted preparation and purification of goblet cell antigen possible.

Therefore, in the context of this invention, goblet cell antigen is provided, which is obtainable by expression by the cell line HT29-18N2 which has differentiated to goblet cells in protein-free medium, as disclosed, e.g., in WO8802774 or WO9808934, or in PFHM II medium (Protein Free Hybridoma Medium, Invitrogen), and which is detectable with anti-goblet cell antibodies from colitis ulcerosa patients.

The goblet cell antigen can be expressed, e.g., by the cell line HT29-18N2 which has differentiated to goblet cells. This cell line has already been used many times in research and is therefore readily obtainable, e.g. from T. Phillips (University of Missouri, Columbia, USA), Euroimmun (Euroimmun A G, Seekamp 31, 23560 Lübeck, Germany), B. L. Rodríguez (Departamento de Genética, CNIC, Havanna, Cuba), R. A. Finkelstein (University of Missouri, Columbia, USA), J. A. Benitez (Morehouse School of Medicine, Atlanta, USA), A. Frey (Forschungszentrum Borstel, 23845 Borstel, Germany), D. Louvard (Institut Pasteur, 75724 Paris Cedex 15, France), R. J. Nijman (Erasmus Medical Center, 3000 D R Rotterdam, The Netherlands), K. Kobayashi (Veterans Affairs Medical Center, Denver, USA) or T. Hibi (School of Medicine, Keio University, Japan).

A culture of HT29-18N2 cells in PFHM II (Protein Free Hybridoma Medium II, Invitrogen) is particularly suitable for differentiation to goblet cells which express the goblet cell antigen. The cells can also be seeded in protein-containing medium (e.g. DMEM (Dulbecco's Modified Eagle's Medium), 10% (v/v) fetal calf serum (FCS), and the change to PFHM II leads to a change in morphology to that of goblet cells. The cells are cultured for at least 2 days, particularly preferably at least 4, 7, 10 or 14 days in the absence of protein.

The cell line HT29-18N2 can be obtained by differentiation of the cell line HT29 in the absence of glucose by the method of Huet et al. (Huet et al., 1987, ibid).

The HT-29 cells are a cell line of a human colon adenocarcinoma, which can be obtained e.g. from DSMZ (Deutsche Sammlung für Mikroorganismen und Zellkulturen GmbH, deposition number ACC299). The adherent, epitheloid cells grow in multiple layers and in large colonies. The development of these non-differentiated precursors of intestinal wall cells can be influenced by the nature of the culture. If glucose in the medium is replaced by galactose, the cells differentiate to enterocytes (approx. 90%) and goblet cells (approx. 10%) in a period of time of from approx. 1 to 2 weeks (Huet et al., 1987, ibid). Further subclones with the properties of the HT29-18N2 clone, that is, also expression of the goblet cell antigen, can be obtained from the parent cell line by the method demonstrated in the publication by Huet et al. Goblet cell antigen can also be obtained from such cell lines under suitable culture conditions.

It has furthermore been found that daughter cell lines (HT29-MTX, HT29-FU, HT29-5M21, HT29-5F7) with some of the properties of goblet cells, as, e.g., the production of mucins typical of goblet cells, can be obtained from the cell line HT29 by culture in the presence of methotrexate and/or 5-fluorouracil (Lesuffleur et al., 1991, Int J Cancer, 49: 731-737, Leteurtre, 2004, Biol Cell, 96, 145-151). Goblet cell antigen can also be obtained from these or analogously obtained cell lines using suitable culture conditions.

The goblet cell antigen is particularly suitable for diagnosis of colitis ulcerosa, since it is recognized specifically by antibodies from the serum of approx. 28% of colitis ulcerosa patients. Since not all colitis ulcerosa patients have antibodies against goblet cell antigen (Stöcker et al., 1987, ibid), for identification of the goblet cell antigen according to the invention it is necessary to use either sera from colitis ulcerosa patients which have tested positively for reactivity with goblet cell antigen, e.g., on primate intestine (see e.g. Example 1) or antibodies from a sufficient number of colitis ulcerosa patients, if appropriate in purified form. Anti-goblet cell antibodies from colitis ulcerosa patients correspond to anti-goblet cell-positive serum from colitis ulcerosa patients. Where appropriate, antibodies from serum which has tested positive can be purified before use as a positive control, e.g. via a protein A or G column.

The goblet cell antigen according to the invention is preferably not detectable with antibodies from Crohn's disease patients. It is furthermore preferably also not detectable with antibodies from patients with other autoimmune diseases (apart from colitis ulcerosa). In the studies carried out to date, no cross-reactivity of antibodies from patients with Crohn's disease or other autoimmune diseases with the goblet cell antigen was found. The presence of antibodies against the goblet cell antigen is thus specific to colitis ulcerosa.

It was possible to demonstrate that both the cells and the culture supernatant contain the goblet cell antigen. It is therefore partly located extracellularly. These results explain the localization of the immunofluorescence, e.g. in primate intestine, with which a cloud-like, blurred fluorescence can be seen in a plane above the goblet cells (Stöcker et al., 1987, ibid). The location within the cells can be illustrated only poorly by this method, since the associated fluorescence is masked by the overlying fluorescence. Nevertheless, the localization can be demonstrated with a confocal laser scanning microscope which is capable of fading out the interfering fluorescence.

In a particularly preferred embodiment, the goblet cell antigen is expressed by the cell line HT29-18N2 which has differentiated to goblet cells in PFHM II. Preferably, the antigen is purified, in particular isolated, and is purified from the cell culture supernatant of the cell line HT29-18N2 which has differentiated to goblet cells in PFHM II.

A particularly simple, but adequate method for purifying the antigen is separation of the culture supernatant from the cells. The purification can be improved by removal of small proteins and low molecular weight constituents of the medium, such as, for example, biotin, e.g. by ultrafiltration or gel filtration. Preferably, the antigen is separated from these constituents by ultrafiltration with a filter having an exclusion size of at least 5 kDa, in particular of at least 100 kDa, and remains in the retentate.

Of course, further purification of the antigen can be carried out, e.g., by chromatography. Preferably, the antigen is separated from other substances by ion exchange chromatography, particularly preferably by an anion exchange chromatography at a pH of ≧5.0, preferably at a pH of ≧7.5, by elution with at least 150 mM NaCl, preferably by elution with at least 200 mM NaCl.

Goblet cell antigen purified via anion exchange chromatography has been characterized by various methods. In an investigation by SDS-PAGE and western blotting with anti-goblet cell-positive serum from colitis ulcerosa patients, only an individual band, which lies above the top band of the marker (185 kDa), manifests itself under non-reducing conditions in the goblet cell antigen-positive fractions. The goblet cell antigen thus preferably has, measured in non-reducing SDS-PAGE, an apparent molecular weight which is greater than 185 kDa.

Under reducing conditions, on the other hand, the sera do not react with the goblet cell antigen. The epitopes recognized in the target antigen are therefore probably mainly conformational in nature, since they seem to be at least partly dependent on the three-dimensional structure of the antigen and/or contain disulfide bridges.

The results furthermore show that the goblet cell antigen has a very high molecular weight. In order to be able to show proteins of this size in differentiated form, agarose gel electrophoreses with a separating range of from approx. 100 to ≧2,000 kDa had to be carried out instead of the polyacrylamide gel electrophoreses conventional in protein biochemistry.

Under these conditions, the mobility of the goblet cell antigen is below that of human IgM (non reduced, molecular weight approx. 950 kDa, L. Stryer, Biochemie [Biochemistry], Spektrum-Verlag, 4th edition 1995, p. 395). However, the mobility is above that of the non-reduced mucin MUC5AC. The apparent molecular weight of the non-reduced goblet cell antigen in agarose gel electrophoresis is thus between 950 kDa and that of the non-reduced MUC5AC (>2,000 kDa, see J. Bara et al., Biochem J, 15, 185-193). The apparent molecular weight can therefore be estimated at greater than 1,000 kDa.

In the preparation, the goblet cell antigen appears to make up the majority of the protein present. Two further protein bands are detectable, one of the contaminating proteins being the mucin MUC5AC, which is recognized by a monoclonal anti-MUC5AC antibody. The other contaminating protein appears to have an apparent molecular weight corresponding to that of non-reduced IgM (approx. 950 kDa) in the non-reduced state, since, in agarose gel, it has a mobility similar to that of non-reduced human IgM.

In a western blot using lectins, a protein of the same mobility was bound by various lectins. Here also, the conclusion that this is the goblet cell antigen is suggested. The goblet cell antigen is therefore preferably detectable with at least one or all of the lectins PHA-L (Phaseolus vulgaris leukoagglutinin), PHA-E (Phaseolus vulgaris erythroagglutinin), RCA (Ricinus communis agglutinin), Con A (concanavalin A), LCA (Lens culinaris agglutinin), PSA (Pisum sativum agglutinin) and AAL (Aleuria aurantia lectin). Under the conditions used, however, the goblet cell antigen was not detectable with the lectins SNA (Sambucus nigra lectin), HHL (Hippeastrum hybrid lectin), MAL I (Maackia amurensis lectin I), SBA (soybean agglutinin), DBA (Dolichus biflorus agglutinin), UEA I (Ulex europaeus agglutinin), SJA (Sophora japonica agglutinin), PNA (peanut agglutinin), WGA (wheat germ agglutinin), GSL I (Griffonia simplicifolia lectin I), PTL I (Psophocarpus tetragonolobus lectin I), WGA s+b (wheat germ agglutinin, succinylated and biotinylated). On the other hand, the contaminating proteins from the anion exchange chromatography all appear not to react with the lectins investigated.

The specificity of the lectins which bind to the goblet cell antigen shows that the antigen comprises galactose, glucose, mannose, N-acetyl-galactosamine and/or fucose (see Table 1, below). However, the present invention also provides non-glycosylated or only partly glycosylated goblet cell antigen which is bound by anti-goblet cell-positive serum.

In contrast to the anti-goblet cell-positive serum, after reduction of the goblet cell antigen the lectins also bind to the glycans which are uninfluenced by the reduction. The mobility of the constituents detected is increased significantly compared with the non-reduced goblet cell antigen, and the band with the previous mobility disappears completely on reduction.

It can therefore be assumed that the goblet cell antigen comprises several peptide chains which are bonded to one another with disulfide bridges in the native state. In a reducing SDS-PAGE, it was therefore possible to carry out a determination of the apparent molecular weight of glycosylated proteins which appear to be constituents of the goblet cell antigen. This indicates that the goblet cell antigen comprises one or more glycosylated proteins of molecular weight 52-54, 56, 66 and/or 80 kDa.

By preparation of the non-reduced total protein from an agarose gel at the position of the band which becomes visible after detection with anti-goblet cell-positive serum, subsequent reduction followed by SDS-PAGE and western blotting with the lectins which bind to the goblet cell antigen, it becomes clear that only proteins having apparent molecular weights of 56, 66, and/or 80 kDa are to be found at this position. In contrast, the proteins in the range corresponding to 52-54 kDa are not present. It can be concluded from this that the goblet cell antigen comprises one or more proteins of the apparent molecular weights 56, 66, and/or 80 kDa.

After cleavage with N-glycosidase F, the apparent molecular weight of these constituents was reduced to a small degree, but the reactivity with the corresponding lectins was retained. From this finding it can be deduced that the goblet cell antigen comprises N- and O-glycosidically bonded glycans. Furthermore, the carbohydrates detectable via the lectins, which are partly terminal carbohydrates, can in some cases be removed by specific glycosidases and the underlying carbohydrates are exposed. In a preferred form, the glycans of the goblet cell antigen are therefore partly or completely removed by chemical or enzymatic methods.

Further purification of the goblet cell antigen, e.g. from the positive fractions of the anion exchange chromatography, is possible by standard methods, for example size fractionation by means of gel filtration or HPLC.

However, purification with the aid of affinity chromatography with antisera from the blood from colitis ulcerosa patients or with the aid of the lectins which bind to the goblet cell antigen is particularly preferred. For the person skilled in the art, further methods result for the purification and isolation of the goblet cell antigen from the culture supernatant or from cells, such as, for example, filtration, chromatography, electrophoresis and centrifugation methods or combinations of these. Such methods of purification of proteins are described e.g. in Lottspeich and Zorbas (Bioanalytik [Bioanalysis], 1998, Spektrum akademischer Verlag, Heidelberg, Berlin) or Sambrook et al. (Molecular cloning: a laboratory manual, 2000, Cold Spring Harbor Laboratory Press, Cold Spring Harbor, N.Y.). The present invention therefore also provides goblet cell antigen which is purified or isolated by such methods and is bound by anti-goblet cell-positive serum.

With the aid of the purified antigen, the DNA sequence which codes for it can be determined by known methods. Thus, e.g., sequencing of the purified protein fragments of the goblet cell antigen allows synthesis of degenerated primers with which the cDNA which codes for goblet cell antigen can be identified in a cDNA library, e.g., from the HT29-18N2 cells cultured in PFHM II. Screening of a cDNA expression library with antibodies generated against goblet cell antigen or with antibodies from colitis ulcerosa patients or the search in suitable databanks is also possible.

Cloning of the cDNA into an expression vector allows recombinant expression of the antigen. Expression of the protein is possible in various systems, e.g. bacterial systems, such as Escherichia coli or Bacillus subtilis, in yeasts, such as Saccharomyces cerevisiae or Pichia pastoris, or other eukaryotic cells, such as 293, CHO, COS or insect cells.

Expression in eukaryotic cells, preferably in a eukaryotic cell line, is preferred, since, here, a glycosylation of the protein and necessary further processing steps can take place. Various expression vectors are known for the particular systems.

In a preferred embodiment, the goblet cell antigen is expressed as a fusion protein, e.g. as Ig fusion protein or using a tag for protein purification, e.g. a polyhistidine tag (His tag). Accordingly, in a preferred embodiment, a recombinant antigen is used. The mentioned methods are described, e.g., in Lottspeich and Zorbas (Bioanalytik [Bioanalysis], 1998, Spektrum akademischer Verlag, Heidelberg, Berlin) or Sambrook et al. (Molecular cloning: a laboratory manual, 2000, Cold Spring Harbor Laboratory Press, Cold Spring Harbor, N.Y.).

In a particularly preferred embodiment, only the parts of the antigen to which the antibodies from colitis ulcerosa patients bind are expressed, in order to avoid, e.g., non-specific binding of antibodies from patients with other autoimmune diseases or to optimize the expression of the antigen. Such parts can comprise either individual reactive regions of the antigen, or fusion proteins from several reactive regions of the antigen or fusion proteins from one or more reactive regions of the antigen, and sequences which do not belong to the goblet cell antigen.

The present invention also provides a monoclonal antibody which recognizes goblet cell antigen. A preferred method for obtaining such an antibody is immunization of a mouse with purified goblet cell antigen. After approx. 2 weeks, the spleen is removed and the spleen cells are fused with myeloma cells in the presence of polyethylene glycol (PEG). The myeloma cells lack the enzyme HPRT, so they cannot survive in HAT selection medium.

Only myeloma cells fused with spleen cells survive culture in the selection medium. The hybridoma cells formed are cloned and tested for the production of antibodies with the aid of the method according to the invention. The method for the preparation of monoclonal antibodies is described in detail, e.g., in Sambrook et al. (Molecular cloning: a laboratory manual, 2000, Cold Spring Harbor Laboratory Press, Cold Spring Harbor, N.Y.).

A particularly preferred method comprises humanization of the monoclonal antibody obtained in this way. This is effected by isolating the associated cDNA from the hybridoma cells and exchanging the sequence which codes for the F_{c} part of the monoclonal antibody by a corresponding human sequence. The hybrid sequence formed in this way can be transferred into a suitable expression vector and expressed in a suitable expression system and purified from this by known methods. In a preferred embodiment, a recombinant antibody is accordingly used. The methods mentioned are described, e.g., in Lottspeich and Zorbas (Bioanalytik [Bioanalysis], 1998, Spektrum akademischer Verlag, Heidelberg, Berlin) or Sambrook et al. (Molecular cloning: a laboratory manual, 2000, Cold Spring Harbor Laboratory Press, Cold Spring Harbor, N.Y.).

A further preferred method for obtaining a monoclonal antibody according to the invention is isolation of specific human B lymphocytes from inflamed intestinal tissue from colitis ulcerosa patients with anti-goblet cell-positive serum. Such lymphocytes can be obtained by excision of the intestinal region affected and culture of the cells present, in particular the B lymphocytes, in a suitable medium. Further isolation of specific B lymphocytes can take place by sorting the cells obtained in a fluorescence-assisted cell sorter (FACS) with the aid of a B lymphocyte-specific antibody labeled with a fluorophore, e.g. anti-CD20, for identification of B lymphocytes, and/or of the purified goblet cell antigen labeled with a second fluorophore, for identification of specific B lymphocytes. These can be converted and selected, e.g., in a manner analogous to the method described above, into hybridoma cells.

An alternative possibility is the establishment of a phage display library based on the cDNAs of the antibodies from the cells obtained in this way. After screening for binding to the goblet cell antigen purified on the basis of the method according to the invention, a specific monoclonal antibody can be obtained by isolating the cDNA which codes for the specific immunoglobulin, conversion of the cDNA into a suitable expression vector, expression in a suitable expression system and purification of the antibody by known methods. In a preferred embodiment, a human recombinant antibody is accordingly used. The methods mentioned are described e.g. in Eggena et al., 1996, J Immunol, 156: 4005-4001.

A further preferred method for obtaining a monoclonal antibody according to the invention is screening of a phage display library based on randomized cDNAs of human antibodies, e.g. the HuCAL Gold Library from MorphoSys, for binding to the purified goblet cell antigen with one of the methods according to the invention. By subsequent transfer of the associated cDNAs into a suitable expression vector, expression in a suitable expression system, purification of the antibody by known methods and screening for desired properties, a Fab fragment specific for the goblet cell antigen can be obtained first. Accordingly, in a preferred embodiment, a human recombinant Fab fragment is used. This can be modified by complementation with a cDNA which codes for the F_{c} part, to give a complete human immunoglobulin.

Complete immunoglobulins can also be obtained directly in an analogous manner using other libraries. The methods mentioned are described in the technical description of the HuCAL Gold Library of MorphoSys.

Further methods for preparation of monoclonal antibodies are evident for the person skilled in the art. The present invention therefore also provides monoclonal antibodies which are prepared by such methods and bind to goblet cell antigen which is bound by anti-goblet cell-positive serum.

Furthermore, a kit for diagnosis of inflammatory bowel diseases which comprises goblet cell antigen and, e.g., instructions for diagnosis of inflammatory bowel diseases is provided in the context of the invention. Preferably, the kit furthermore also comprises the monoclonal antibody, so that positive sera from colitis ulcerosa patients do not have to be used for carrying out the method according to the invention.

A method for detecting antibodies against goblet cell antigen, in which a biological sample is contacted with an antigen and binding of the antibody to the antigen is detected, is also provided in the context of this invention, the antigen being obtainable by expression by the cell line HT29-18N2 which has differentiated to goblet cells in PFHM II and being detectable with anti-goblet cell antibodies from colitis ulcerosa patients. The goblet cell antigen described above is employed for the method.

The method is particularly suitable for diagnosis of colitis ulcerosa, since the antigen is specifically recognized by antibodies from the serum of approx. 28% of colitis ulcerosa patients. The sample which is to be analyzed for antibodies against goblet cell antigen is a biological sample, in general a blood or serum sample.

Preferably, it is a sample from a patient with an inflammatory bowel disease, in whom colitis ulcerosa could be present. However, it can also be e.g. the supernatant of a hybridoma or a phage display library.

In the method according to the invention, various methods can be employed for detecting the binding of the antibodies to the antigen. This is preferably carried out with an ELISA or blot, e.g. western blot or dot blot.

Moreover, rapid tests for qualitative detection of antibodies for reading a color reaction on a test strip can also be used. Such tests are in general based on a sandwich solid phase immunoassay, in which goblet cell antigen is coupled to a test field or in a line on the test strip. The test strip is brought into contact, e.g., with a blood sample or diluted patient serum, which possibly contains anti-goblet cell antigen antibodies. Secondary anti-human immunoglobulin antibodies of suitable specificity, e.g. conjugated with gold conjugate or with an enzyme, such as alkaline phosphatase or horseradish peroxidase, collect in the test field only if the sample contained antibodies. If appropriate, the result must also be rendered visible with a chromogenic substrate.

The present invention furthermore provides a method for detection of antibodies against goblet cell antigen, in which a sample is contacted with a cell line which is derived from the HT29 cell line and has differentiated to goblet cells in protein-free medium, and the binding of antibodies to the antigen is detected. The culture conditions which are preferably used for this have already been described in detail above. The goblet cell antigen according to the invention expressed by the cells under these culture conditions specifically reacts with anti-goblet cell antibodies from colitis ulcerosa patients.

It is thus not detectable with antibodies from Crohn's disease patients, and preferably also not with antibodies from patients with other autoimmune diseases. Preferably, the cell line which has differentiated to goblet cells in protein-free medium is the cell line HT29-18N2.

Carrying out the method according to the invention with the cells described allows detection of the binding of the antibodies to the antigen with indirect or direct immunofluorescence. This method is currently the standard method for detecting goblet cell antigen (Stöcker et al., 1984, 1987, ibid) using intestinal tissue, so that a comparison of the methods is particularly easily possible. This comparison shows that with anti-goblet cell-positive sera, the cells show a cloud-like, blurred fluorescence in a plane above the cells which is very similar to the reaction of the same sera on primate and human fetal intestine.

By comparison, blood donor sera showed no specific reactivity. A comparison of the sensitivity of the substrates in indirect immunofluorescence shows that the HT29-18N2 cell line is more suitable than primate intestine as a substrate for diagnostics for detecting anti-goblet cell antibodies. Non-specific reactions are absent or are negligibly low, so, at any time, results which are comparable and can be evaluated unambiguously can be achieved in the diagnosis of colitis ulcerosa with the indirect immunofluorescence test with HT29-18N2 cells as the substrate.

The prevalence of antibodies against goblet cell antigen determined hitherto with the method according to the invention is approx. 28%. This is in agreement with the values already determined by Stöcker et al. (1987, ibid). Diseases with proven autoimmune pathogenesis also show a prevalence of the corresponding autoantibodies of less than 90% in most cases.

The relatively low percentage of anti-goblet cell antibodies with colitis ulcerosa could be caused by a neutralization of the circulating antibodies by erythrocytes or other cells or structures: It has, e.g., been found that patients with blood group 0 show a prevalence of only 19%, while in patients with blood group AB it is 66%.

The predictive value of a detection of antibodies against goblet cell antigen is 100%. A method for diagnosis of inflammatory bowel diseases in which these antibodies are detected with the method according to the invention is therefore provided in the context of the invention, wherein a colitis ulcerosa is diagnosed by detection of antibodies against goblet cell antigen.

Reliable test systems have already been established for anti-pancreas, anti-Saccharomyces cerevisiae antibodies and pANCA, and the present invention provides such a test system for anti-goblet cell antigen. A combination of the four tests would in many cases make endoscopy superfluous. In approx. 80. % of patients with chronic inflammatory bowel diseases, a differential diagnosis can thus already be made serologically by investigation of the antibodies against goblet cell antigen, pancreas secretion, Saccharomyces cerevisiae and pANCA.

The present invention also provides the use of goblet cell antigen for the preparation of a pharmaceutical composition for treatment of colitis ulcerosa. The present invention furthermore provides a pharmaceutical composition, preferably for treatment of colitis ulcerosa, which comprises goblet cell antigen, optionally with a pharmaceutically suitable carrier agent and/or auxiliaries.

In the treatment of colitis ulcerosa, for example, a body fluid of the patient is brought into contact with the antigen.

In the context of this invention, body fluid is understood as meaning, e.g., blood or intestinal content or the secretion of the mucous membrane of the patient. Preferably, goblet cell antigen is coupled to an inert matrix, e.g., silica gel or a carbohydrate which is indigestible by humans, such as alginate, cellulose, pectin, carrageen or the like. This conjugate is then brought into contact with a body fluid of a patient with anti-goblet cell antibodies in order to bind the anti-goblet cell antibodies contained in this body fluid to the conjugate.

There are various possibilities for bringing the body fluid of a patient into contact with such a matrix. One possibility is haemodialysis to remove the antibodies. In this context, blood of a colitis ulcerosa patient is taken from the body and brought into contact with the conjugated matrix. The blood is then separated from the conjugate and returned, so that the anti-goblet cell antibodies are removed from the body fluid and can no longer participate in the inflammatory process. Since most of the anti-goblet cell antibodies are present as IgG, removal of the specific antibodies of this isotype present in the blood significantly reduces the total amount of antibodies. The present invention therefore also provides the use of goblet cell antigen for ex vivo removal of anti-goblet cell antibodies from the blood of colitis ulcerosa patients.

In addition to such a contact ex vivo, an in vivo contact is also possible. For example, an inert matrix with coupled goblet cell antigen can be brought into the intestine through the stomach in tablet form, that is to say in a formulation resistant to gastric juice.

Coupling of a cytotoxin to the goblet cell antigen is furthermore preferred. After administration to colitis ulcerosa patients with anti-goblet cell antibodies, the conjugate binds to the specific B lymphocytes and destroys these or induces apoptosis in them. Preferably, the cytotoxin is specific for B lymphocytes.

Alternatively, a pharmaceutical composition which comprises the goblet cell antigen can also be used for inducing tolerance to the goblet cell antigen. Preferably, for this, the goblet cell antigen is formulated with antiinflammatory cytokines, such as e.g. TGF-β or IL-10, which can induce regulatory T cells (Chen et al., 2003, J. Exp. Med., 198, 1875-86). Suitable adjuvants and further methods for inducing tolerance are known in the prior art, e.g. blocking co-stimulating signals or administration of the antigen in the context of tolerogenic autologous dendritic cells (Xiao et al., 2003, BioDrugs, 17, 103-11).

The following experiments show particular embodiments of the invention. It was first attempted to employ human or primate intestinal tissue for isolation of goblet cell antigen. As already known in the literature, this tissue is suitable for detection of anti-goblet cell antibodies by means of indirect immunofluorescence. However, it was found that with none of the experiments carried out (western blotting, neutralization tests, lectin ELISA), specific reactions of one or more fractions of an intestine preparation with the anti-goblet cell-positive sera can be detected. Under the given conditions, intestinal tissue therefore does not appear to be suitable for purification of goblet cell antigen and preparation of standardized test systems based on an ELISA or blotting technique. Analogous results were achieved with the cell line HT29 cultured under standard conditions (see information on the cell line of the Deutsche Sammlung für Mikroorganismen [German Collection of Microorganisms]).

Thus, the cell line HT29-18N2 was subsequently investigated, which was found, surprisingly, to express goblet cell antigen under the culture conditions described above. Both indirect immunofluorescence and ELISA and western blotting were possible with this cell line, so it is suitable for detecting anti-goblet cell antibodies. Furthermore, goblet cell antigen can be purified with the aid of this cell line.

### LEGEND TO THE FIGURES

FIG. 1: Indirect immunofluorescence with intestinal tissue

A: Representation of autoantibodies from colitis ulcerosa patients against intestinal goblet cells (anti-goblet cell antibodies) by indirect immunofluorescence on human fetal intestine (200-fold magnification).

B: Representation of autoantibodies against intestinal goblet cells (FITC-labeled) and anti-MUC2 antibodies (TRITC-labeled) by indirect immunofluorescence on prim. adult intestine (200-fold magnification).

C, D: Representation of the anti-MUC2 antibody by indirect immunofluorescence in prim. adult intestine (C: 200-fold magnification, D: 400-fold magnification).

FIG. 2: Fractionation of intestine tissue

A: Silver gel of the fractions of a preparation from human adult intestinal tissue. In each case 1 μl of the stated fractions was applied. The samples were obtained by centrifugation at increasing revolutions per minute.

B, C: Western blot of the fractions of the intestinal preparation, incubated with a mixture of anti-goblet cell-positive sera (B) and blood donor sera (C) (1: homogenate, 2: supernatant 1, 3: sediment 1, 4: supernatant 2, 5: sediment 2, 6: supernatant 3, 7: sediment 3, 8: supernatant 4, 9: sediment 4, 10: supernatant 5, 11: sediment 5).

FIG. 3: Diagram of the structure of a lectin ELISA

The biotinylated lectins bind to a plate coated with streptavidin and blocked with BSA. Antigens present in the sample are bound via their carbohydrate content and a reaction with primary antibodies from the serum and POD-labeled secondary antibodies from the conjugate is specifically detected.

FIG. 4: Indirect immunofluorescence with HT29-18N2 cells

A, B: Representation of autoantibodies against goblet cells (A), of a blood donor serum (B), of anti-MUC2 antibodies (C) and of anti-MUC5AC antibodies (D) by indirect immunofluorescence on HT29-18N2 cells after differentiation to goblet cells in PFHM II medium (200-fold magnification).

FIG. 5: Analysis of HT29-18N2 cell culture supernatants

A: Representation of the reactivity of anti-goblet cell-positive sera (serum 1 to 3) compared with blood donor sera (BD 1 to 4) and the anti-MUC5AC antibody by dot blotting.

The samples were concentrated with a filter having an exclusion size of 100 kDa and the medium was replaced by PBS. In each case 2 μl of sample were applied. The samples were cell culture supernatants from various culture bottles (A, B and C) obtained at different time points after the start of the differentiation.

B: Representation of the reactivity of anti-goblet cell-positive sera and blood donor sera (BD sera) compared with chromatography fractions of an HT29-18N2 cell culture supernatant.

In each case 5 μl of the fraction were applied.

The right-hand strip shows the sequence of fractions applied (A means application). The negative control was incubated with buffer instead of a serum dilution.

FIG. 6: Detection of antibodies against goblet cell antigen by ELISA

A: Reactivity of various starting substrates (cells and cell culture supernatant) with anti-goblet cell antibodies from sera of colitis ulcerosa patients (CU, dark) and blood donor sera (BD, light) in ELISA.

B: Reactivity of the chromatography fractions of a cell culture supernatant with anti-goblet cell-positive and blood donor sera in ELISA.

FIG. 7: Characterization of goblet cell antigen in SDS-PAGE/western blot

A: SDS-PAGE/western blot of the chromatography fractions under reducing/non-reducing conditions

The same amounts of a sample of a chromatography fraction positive for goblet cell antigen were used under reducing (left-hand track each time) and non-reducing (right-hand track each time) conditions for SDS-PAGE. The western blot was stained with anti-MUC5AC antibody (middle strip) and anti-goblet cell-positive serum (right-hand strip). MultiMark was used as the marker.

B: Agarose gel electrophoresis/western blot of the chromatography fractions under reducing/non-reducing conditions

The same amounts of a sample of a chromatography fraction positive for goblet cell antigen were used under reducing (left-hand track each time) and non-reducing (right-hand track each time) conditions for an agarose gel electrophoresis. The western blot was stained with anti-goblet cell-positive sera (1st and 2nd strip from the left), blood donor sera (3rd and 4th strip from the left) and anti-MUC5AC antibody (right-hand strip).

C: FITC labeling of a goblet cell antigen-positive chromatography fraction

The sample of the chromatography fraction was labeled with FITC and analyzed under reducing (lane 2) and non-reducing (lane 3) conditions. A non-labeled, non-reduced sample, detected with anti-goblet cell antigen-positive serum (lane 4) or anti-MUC5AC antibody (lane 5) and non-reduced human IgM (lane 1) serve as a comparison.

FIG. 8: Characterization of the glycans of goblet cell antigen by western blotting with lectins

A: Agarose gel electrophoresis/western blot of the chromatography fraction under non-reducing conditions

Strip 1 (from left to right): anti-goblet cell-positive serum; strip 2 and 3: blood donor sera; strip 4: SNA; strip 5: PHA-L; strip 6: PHA-E; strip 7: HHL; strip 8: RCA; strip 9: MAL I; strip 10: Con A; strip 11: SBA; strip 12: DBA; strip 13: UEA I; strip 14: SJA; strip 15: PNA; strip 16: WGA; strip 17: LCA; strip 18: GSL I; strip 19: PSA; strip 20: PTL I; strip 21: AAL; strip 22: WGA s+b.

B: Agarose gel electrophoresis/western blot/lectin blot of the chromatography fraction under reducing conditions

Strip 1 (from left to right): SNA; strip 2: PHA-L; strip 3: PHA-E; strip 4: HHL; strip 5: RCA; strip 6: MAL I; strip 7: Con A; strip 8: SBA; strip 9: DBA; strip 10: UEA I; strip 11: SJA; strip 12: PNA; strip 13: WGA; strip 14: LCA; strip 15: GSL I; strip 16: PSA; strip 17: PTL I; strip 18: AAL; strip 19: WGA s+b.

C: SDS-PAGE/western blot/lectin blot of the chromatography fraction under reducing conditions

Strip 1 (from left to right): SNA; strip 2: PHA-L; strip 3: PHA-E; strip 4: HHL; strip 5: RCA; strip 6: MAL I; strip 7: Con A; strip 8: SBA; strip 9: DBA; strip 10: UEA I; strip 11: SJA; strip 12: PNA; strip 13: WGA; strip 14: LCA; strip 15: GSL I; strip 16: PSA; strip 17: PTL I; strip 18: AAL; strip 19: WGA s+b.

D: Characterization of the goblet cell antigen after preparation from an agarose gel

left-hand track: marker; right-hand track: goblet cell antigen after preparation from an agarose gel, lectin blot with PHA-E.

FIG. 9: Characterization of the glycans of goblet cell antigen after deglycosylation

A, B: Lectin blot after deglycosylation with N-glycosidase F

left-hand track: sample of the chromatography preparation after N-glycosidase digestion; right-hand track: sample of the chromatography preparation without digestion, in each case the same amounts of the sample.

A: lane 1: marker; lane 2: N-glycosidase F; strip 1: PHA-L; strip 2: PHA-E; strip 3: RCA; strip 4: LCA.

B: lane 1: marker; lane 2: N-glycosidase F; strip 5: PSA; strip 6: AAL.

### EXAMPLES

### Example 1

### Identification of Goblet Cell-Positive Sera from Colitis Ulcerosa Patients

Detection of the antigen to be sought is possible by using anti-goblet cell-positive sera. Sera from colitis ulcerosa patients are therefore investigated in immunofluorescence for their reactivity with goblet cells with the aid of slides with sections of adult primate intestine, human fetal intestine (from excess material of pathologically analyzed samples) and human adult intestine (from surgical resection material). For the investigation e.g. slides from EUROIMMUN together with associated auxiliary reagents are used.

The test is carried out by standard methods, e.g. in accordance with the instructions for the indirect immunofluorescence test of EUROIMMUN, wherein conjugates of classes IgG and IgA are exclusively employed. 200 sera from colitis ulcerosa patients are analyzed in this manner, and anti-goblet cell-positive sera can be employed as a positive control for further experiments.

### Results:

Anti-goblet cell-positive sera show a cloud-like, blurred fluorescence in a plane above the goblet cells. In addition, a non-specific background fluorescence can be seen with many sera. Blood donor sera and samples which react negatively show this background fluorescence only, with a different intensity depending on the serum. Sera from colitis ulcerosa patients which react to goblet cell antigen in this test are referred to as anti-goblet cell-positive sera.

In a similar manner to that already demonstrated by Stöcker et al. (1987, ibid), 28% of the sera analyzed were anti-goblet cell-positive, i.e. they led to the fluorescence typical of goblet cell antigen.

### Example 2

### Staining of Goblet Cell Antigen in Intestinal Tissue

Tissue sections from primate intestine, human fetal and adult intestine were investigated for goblet cell antigen in indirect immunofluorescence with anti-goblet cell-positive sera, as described in Example 1. Expression of further antigens was investigated.

For detection of the additionally employed monoclonal antibodies against the mucins MUC2 (Abcam Ltd) and MUC5AC (NeoMarkers, Inc.), FITC (fluorescein)-labeled anti-mouse antibodies of class IgG are employed as conjugates, and for the anti-MUC3 antibodies (Quartett GmbH) FITC-labeled anti-rabbit antibodies, also of class IgG, are employed. In the case of double fluorescence, the slides are washed after serum incubation and then incubated with one of the monoclonal antibodies for 30 minutes at room temperature. After the subsequent washing step, a mixture of anti-human IgG-FITC (diluted 1:5 in PBS-Tween) and anti-mouse IgG-TRITC (tetramethylrhodamine isothiocyanate) (diluted 1:200 in PBS-Tween) is employed as conjugate.

### Results:

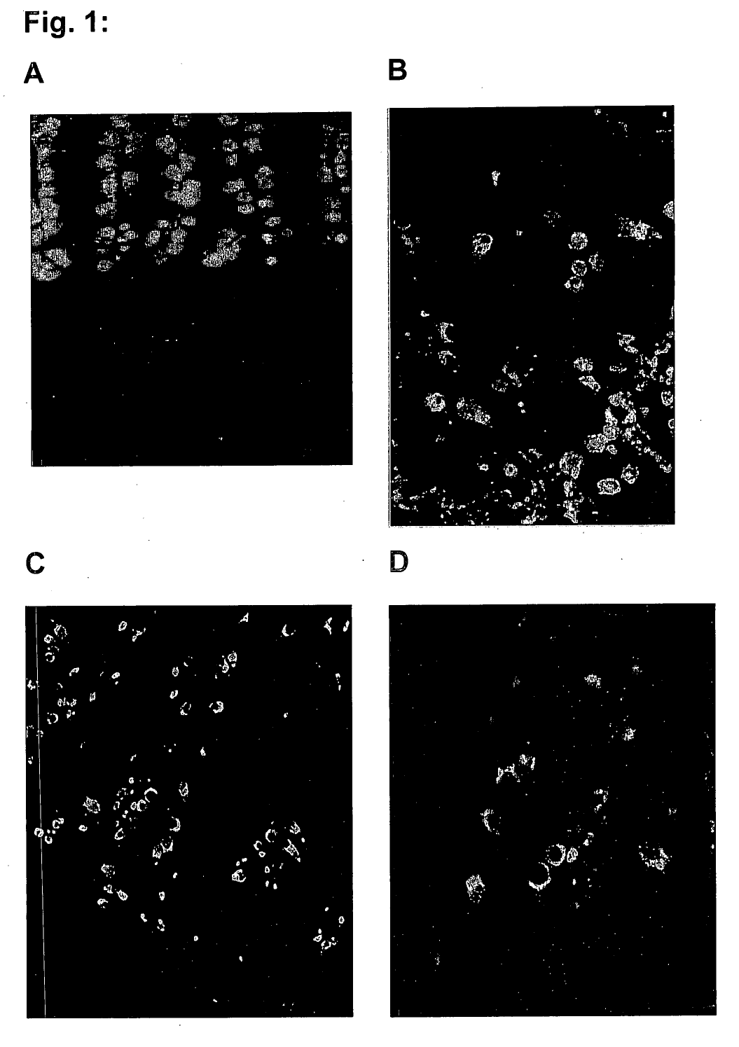

The fluorescence typical of goblet cell antigen manifests itself with the anti-goblet cell-positive sera on all the intestine sections employed (FIGS. 1A and B). The additionally tested monoclonal antibodies against the mucins 2, 3 and 5AC, which are all expressed in the intestine (Hayashi et al., 2001, Digestion 63, 28-31) show different patterns and localizations.

The anti-MUC2 antibody shows a granular fluorescence which is limited to the cytoplasm of the goblet cells (FIGS. 1C and D). After incubation with the anti-MUC3 antibody, other intestinal structures also react, apart from the goblet cells. The anti-MUC5AC antibody, which is the only one of the monoclonal antibodies used which reacts with the completely glycosylated antigen, according to the manufacturer, like the anti-MUC2 antibody shows a goblet cell-specific fluorescence pattern, the pattern after incubation with the anti-MUC 5AC antibody being similar to that after the incubation with goblet cell-positive sera. After incubation with the anti-MUC2 antibody, however, in addition to a fluorescence located in the cytoplasm, a membrane-associated fluorescence is also manifested. The localization of the positive signal can be illustrated with the aid of double fluorescence with an anti-goblet cell-positive serum and the goblet cell-specific anti-MUC2 antibody. The anti-MUC2 antibody detected via TRITC-labeled anti-mouse antibodies reacts—as described—with the cytoplasm of the goblet cells, and the goblet cell-specific reaction of the serum rendered visible with FITC-labeled anti-human antibodies is in the plane above this (FIG. 1B).

### Example 3

### Fractionation of Intestinal Tissue

Human adult intestine is weighed in the frozen state and, on ice, is covered with a layer of about 3 volumes of TNE-T buffer (50 mM Tris-HCl pH 7.4, 150 mM NaCl, 5 mM EDTA, 1 mM PMSF (phenylmethylsulfonyl fluoride), 0.1% Triton X-100). After thawing, the tissue is first cut with scissors and then comminuted with a Miccra D-8 (ART—moderne Labortechnik) three times for 1 minute each time (level C) while cooling with ice. For cooling the sample, a pause of 5 minutes is maintained between each step.

The preparation is centrifuged for 30 minutes at 500×g and 4° C., the supernatant is transferred to a container and the sediment is resuspended in one volume of TNE buffer (50 mM Tris-HCl pH 7.4, 150 mM NaCl, 5 mM EDTA, 1 mM PMSF). Thereafter, a further centrifugation step for 30 minutes at 2,000×g and 4° C. follows. The supernatant is removed again and the sediment is resuspended in one volume of TNE buffer.

The sample is then centrifuged for 30 minutes at 21,250×g and 4° C., the supernatant is removed and the sediment is resuspended in one volume of TNE buffer. If a fatty layer is visible after this step, the suspension is filtered through glass wool.

The preparation is centrifuged for 60 minutes at 100,000×g and 4° C. The supernatant is transferred to a container, the sediment is resuspended in one volume of TNE buffer and the suspension is then centrifuged for 60 minutes at 220,000×g and 4° C. After the supernatant has been transferred to a container, the sediment is resuspended in one volume of TNE buffer.

After the homogenization and subsequent fractionation by ultracentrifugation, the individual supernatants and samples of the particular sediments are investigated by gel electrophoresis (NuPage-System, Invitrogen, using MES buffer) and silver staining.

The samples are analyzed in parallel under non-reducing and reducing (15.6 mM DDT, 1.25% Iodoacetamide) conditions. Marker 12 and MultiMark from Invitrogen are run as markers.

The silver staining is carried with standard methods, e.g., by the method of Heukeshoven (1988, Electrophoresis 9, 28-32).

### Results:

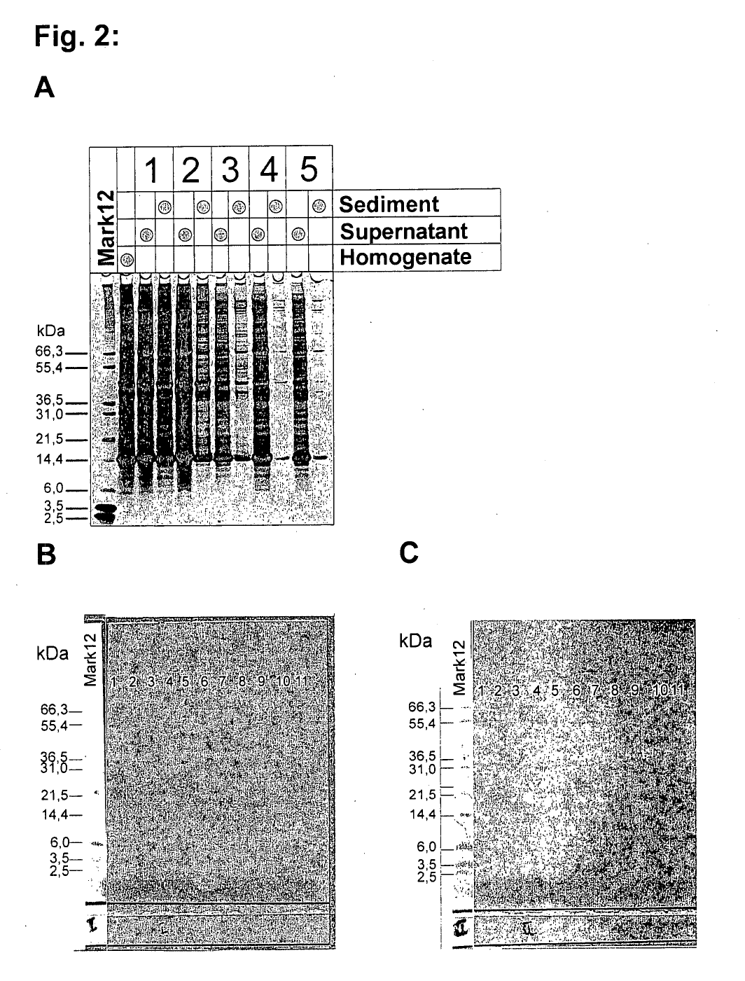

FIG. 2A shows the result of the fractionation. Because of the high protein concentration in the homogenate, which still contains all constituents of the intestinal tissue, individual bands can scarcely be demarcated from one another. The same is true for the supernatants 1 and 2 and the sample of sediment 1. In sediment 2, individual bands can already be seen. From the 3rd centrifugation step, the separating off by the ultracentrifugation is clearly visible, and individual bands can be clearly demarcated from one another. In the sample of sediment 5, only few proteins are still detectable, the molecular weights of these all being above the 14.4 kDa band of the marker. A removal of individual tissue constituents is indeed visible, but no striking features can be seen with respect to the target antigen.

### Example 4

### Analysis of Fractions in the Neutralization Test

The dilution at which the corresponding anti-goblet cell-positive serum just still shows a goblet cell-specific reaction, determined by a dilution series in immunofluorescence, is referred to as titer and is employed for a neutralization test with the samples of the intestine preparation.

For the neutralization test, various amounts of fractions of the intestine preparation from Example 3 are added to 100 μl each of an anti-goblet cell-positive serum (diluted in PBS-Tween, so that a limiting titer is achieved, see below) and incubated for 30 minutes at room temperature. The preparations are then centrifuged for 10 minutes at 13,000 rpm in an Eppendorf centrifuge. The supernatant is employed instead of the serum dilution for an indirect immunofluorescence with primary human adult intestinal tissue. A control field which has been incubated only with the starting dilution of the serum serves as a comparison here.

### Result:

No neutralizing action of one of the fractions against an anti-goblet cell-positive serum can be determined in the test. Since all fields of the slide react with the same intensity and identical pattern, no conclusion can be made regarding the fraction in which the target antigen is present. Repeating the test with increasing concentrations of the individual samples in the preparation also leads to an identical result.

### Example 5

### Investigation of Fractions in Western Blot

The fractions of the intestine preparation are to be investigated for the presence of the target antigen with the aid of a specific detection with anti-goblet cell-positive sera in western blotting. After the separation by SDS gel electrophoresis, the proteins are transferred onto a nitrocellulose membrane, e.g., with the wet blotting method by means of a blotting apparatus from the company Hoefer.

After preparation, the samples are applied to SDS-PAGE gels, and, after transfer to nitrocellulose membranes, they are first stained with Ponceau S and then incubated with anti-goblet cell-positive or blood donor sera.

For the Ponceau S staining, the nitrocellulose membrane is covered with Ponceau S solution for 10 minutes, while shaking. After the incubation, the membrane is destained again with distilled water, until the marker and stained proteins are clearly visible as red bands.

For the specific immunological detection with antibodies, the membrane, cut into strips if appropriate, is first blocked for 15 minutes with universal buffer (EUROIMMUN) with 3% (w/v) of powdered milk.

It is then incubated for 3 hours with control or patient sera (1:500 in universal buffer with 3% (w/v) of powdered milk). Washing is then carried out with universal buffer three times for 5 minutes each time. In a second incubation step, the antibodies bound to the proteins in the positive case react with a conjugate solution (EUROIMMUN, diluted 1:10 in universal buffer), which contains anti-human antibodies labeled with alkaline phosphatase. Washing is then carried out as after the serum incubation. In a third incubation step, the bound antibodies are then detected with an NBT/BCIP substrate solution (4-nitroblue tetrazolium chloride/chlorobromoindolyl phosphates, EUROIMMUN).

### Results:

After the Ponceau S staining, a similar pattern of bands can be seen as after silver staining, the intensity of many bands being weaker because of the lower sensitivity of the Ponceau S staining. As with the silver gel, no striking features can be seen.

After the incubation of the membranes with anti-goblet cell-positive or blood donor sera, signals can be seen on both western blots (FIGS. 2B and C). In almost all the samples, a band at approx. 55 kDa reacts particularly clearly, which can also be detected with the Ponceau S staining, although it has a rather weak intensity here. However, a comparison of the two membranes shows that identical patterns of bands result on incubation with anti-goblet cell-positive sera and sera from blood donors. These reactions therefore have to be evaluated as non-specific. None of the striking bands of the Ponceau S staining reacts with the sera in a detectable intensity. By this test also, no conclusion can be made regarding the presence of the target antigen in one of the fractions, since no anti-goblet cell-specific reaction can be detected.

### Example 6

### Investigation of the Fractions in Lectin ELISA

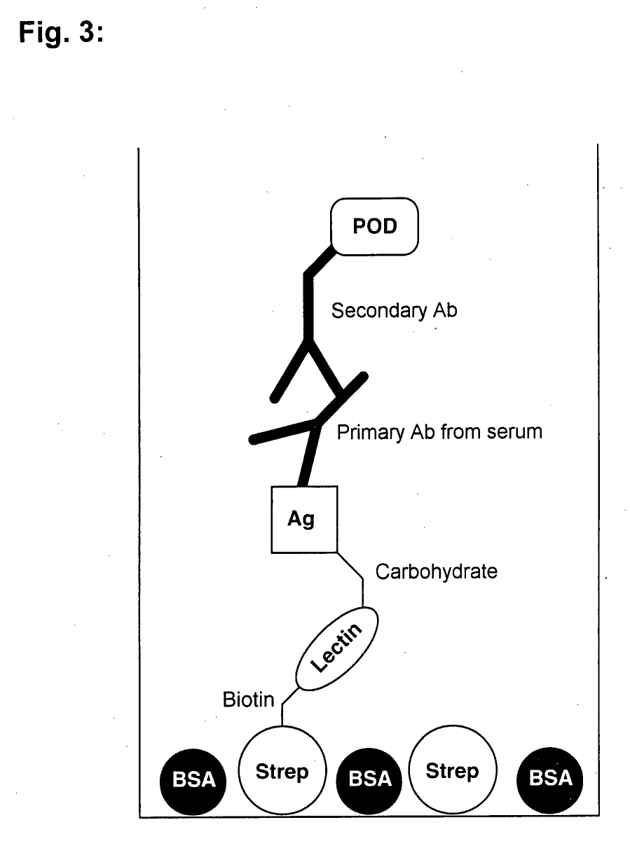

For the lectin ELISA, a MaxiSorb plate from the company Nunc is coated with streptavidin (100 μl of a 0.5 μg/ml solution in PBS, overnight at 4° C. or 2 hours at room temperature) and coated with 100 μl each of lectin dilution (1 μg/ml in PBS, see Table 1) for 30 minutes at room temperature. The plate is then washed (three times with 300 μl of wash buffer (EUROIMMUN) or PBS-Tween each time). An incubation with 100 μl of a dilution of the fractions of the intestine preparation from Example 3 (1:100 in PBS) per well, respectively, for 30 minutes at room temperature then follows. After a washing step, blocking with 200 μl each of 0.5% (w/v) BSA in PBS for one hour at room temperature follows. Thereafter, the plate is washed. For the serum incubation, in each case 100 μl of serum dilution (1:100 in PBS) are incubated for 30 minutes at room temperature. The plate is washed, before incubation is carried out with in each case 100 μl of conjugate solution (EUROIMMUN), which contains peroxidase-labeled anti-human antibodies, for 30 minutes at room temperature. After another washing step, the plate is incubated for 10 minutes with 100 μl of substrate solution (EUROIMMUN) per reaction well at room temperature. At the end of the 10 minutes, the reaction is stopped by addition of 100 μl of stop solution (EUROIMMUN) each, and the extinction at 450 nm is measured with a photometer (Tecan Spectra). The diagram of a lectin ELISA is shown in FIG. 3.

TABLE 1
Selection of biotinylated lectins (Vectorlabs)

| Lectin | Specificity |
|---|---|
| AAL (Aleuria aurantia | 1,3-L-fucose |
| lectin) | |
| Con A (concanavalin A) | α-D-glucose α-D-mannose |
| DBA (Dolichus biflorus | N-acetyl-α-D-galactosamine |
| agglutinin) | |
| GSL I (Griffonia | α-D-galactose |
| simplicifolia lectin I) | N-acetyl-α-D-galactosamine |
| HHL (Hippeastrum hybrid | α-D-mannose |
| lectin) | |
| LCA (Lens culinaris | α-D-glucose α-D-mannose |
| agglutinin) | |
| MAL I (Maackia amurensis | N-acetylneuramic acid-α-2,3- |
| lectin I) | galactose |
| PHA-E (Phaseolus vulgaris | N-acetyl-α-D-galactosamine |
| erythroagglutinin) | |
| PHA-L (Phaseolus vulgaris | N-acetyl-α-D-galactosamine |
| leukoagglutinin) | |
| PNA (peanut agglutinin) | β-D-galactose |
| PSA (Pisum sativum | α-D-glucose α-D-mannose |
| agglutinin) | |
| PTL I (Psophocarpus | galactosamine |
| tetragonolobus lectin I) | |
| RCA (Ricinus communis | β-D-galactose |
| agglutinin) | N-acetyl-α-D-galactosamine |
| SBA (soya bean | N-acetyl-α-D-galactosamine |
| agglutinin) | β-D-galactose |
| SJA (Sophora japonica | N-acetyl-α-D-galactosamine |
| agglutinin) | β-D-galactose |
| SNA (Sambucus nigra | N-acetylneuramic acid-α-2,6- |
| lectin) | galactose |
| UEA I (Ulex europaeus | α-L-fucose |
| agglutinin) | |
| WGA (wheat germ | (N-acetyl-β-(1,4)-D-glucosamine)_{>2} |
| agglutinin) | sialic acids |
| WGA succinylated (wheat | N-acetyl-glucosamine |
| germ agglutinin) | |

### Results:

Analysis of the reactivity of lectins with the samples of the intestine preparation leads to different extinction values, depending on the lectin and fraction. In order to be able to distinguish between specific and non-specific reactions, the preparations are incubated in parallel with sera which have tested anti-goblet cell-positive in the indirect immunofluorescence and sera from blood donors. In the tests carried out, comparable extinction values result with each lectin for both preparations (Table 2). In supernatant 1, e.g., the lectin AAL shows an increased reactivity which, however, has to be regarded as non-specific, since anti-goblet cell-positive and blood donor sera and even the negative control, which was incubated with PBS instead of serum dilution, react to similar degrees. The lectins RCA, PSA, LCA and SNA also show increased reactivities, although these are found both with anti-goblet cell-positive and blood donor sera and with the control. The reactions therefore have to be rated non-specific. Comparable results are also achieved in the testing of the other preparations. No sample of the preparation shows a specific reactivity with one of the lectins available.

TABLE 2
Extinction values (450 nm) of a lectin ELISA with
supernatant 1 of the intestine preparation

| | Anti-goblet | | |
|---|---|---|---|
| Lectin | cell-positive sera | Blood donor | Neg. control |
| UEA I | 0.041 | 0.043 | 0.032 |
| RCA | 0.268 | 0.182 | 0.185 |
| Con A | 0.170 | 0.102 | 0.132 |
| WGA | 0.048 | 0.050 | 0.039 |
| SBA | 0.071 | 0.031 | 0.073 |
| DBA | 0.030 | 0.035 | 0.033 |
| MAL I | 0.025 | 0.037 | 0.025 |
| PNA | 0.028 | 0.024 | 0.022 |
| SJA | 0.036 | 0.044 | 0.022 |
| PHA-L | 0.161 | 0.205 | 0.160 |
| AAL | 0.932 | 0.895 | 1.790 |
| LCA | 0.571 | 0.248 | 0.670 |
| PSA | 0.530 | 0.267 | 0.643 |
| SNA | 0.219 | 0.116 | 0.176 |
| HHL | 0.082 | 0.067 | 0.097 |
| PBS - | 0.023 | 0.037 | 0.019 |
| control | | | |

### Example 7

### Culture of HT29-18N2 Cells

The HT29-18N2 cells are a daughter cell line of HT29 cells. The protocol for the culture and the clone were originally developed by Prof. Dr. Tom Phillips, University of Missouri, Columbia (1988, Gastroenterology 94: 1390-1403).

1.7×10⁴ cells/cm² are seeded in plastic bottles and cultured under standard conditions at 37° C. with 5% CO₂ in Dulbecco's Modified Eagle's medium (DMEM, Invitrogen) with addition of 10% fetal calf serum (FCS), until confluence is reached. The cells are then harvested by addition of trypsin/EDTA and seeded again in DMEgal medium in plastic bottles. Since the change in the medium composition results in a high death rate and a slower growth rate, the medium is changed every day.

DMEgal medium is glucose-free in the sense that it contains only residues of glucose from the FCS, and these are present in a lower amount than galactose. It is composed of 8.3 g DME medium base (Sigma), 0.1% (w/v) galactose (Sigma), phenol red solution (8 mg/ml; Sigma), 0.37% (v/v) NaHCO₃ (Sigma) and 10% (v/v) of dialyzed FCS (JRH Biosciences).

When cultured in DMEM, the cells are highly aggregated and grow in islands in several layers. After seeding in DMEgal, an increased mortality rate is observed. The surviving cells no longer grow in islands, but are seen as individual cells, and demarcated from one another under the microscope.

When confluence is again reached, frozen cultures of a part of the cells are prepared in freezing medium (90% (v/v) FCS and 10% (v/v) DMSO), and are stored in nitrogen.

The remaining cells are seeded on sterile cover glasses (26×76 mm; 7.2×10⁶ cells/cover glass). To achieve a faster growth rate, the DMEgal is replaced by DMEM/10% FCS. After 4 days, the medium is changed from DMEM/10% FCS to DMEgal or protein-free medium, e.g. Protein Free Hybridoma Medium II (PFHM II, Invitrogen).

In parallel, HT29-18N2 cells are cultured in protein-containing DMEM (DMEM/10% FCS). Unless mentioned otherwise, the designation HT29-18N2 cells in the following relates to cells after culture in the protein-free medium PFHM II.

When a uniform and adequate cell density is reached, slides are produced from each half of a cover glass using the biochip technology of EUROIMMUN, and the other half is tested in western blot after breaking down the cells by means of ultrasound in TNE-T buffer (50 mM Tris-HCl pH 7.4, 150 mM NaCl, 5 mM EDTA, 1 mM PMSF, 0.1% Triton X-100).

### Example 8

### Indirect Immunofluorescence with HT29-18N2 Cells

Fixation with Acetone

The cover glass is rinsed twice with PBS. Excess liquid is allowed to drain off on cellulose, before the cover glass is first rinsed in acetone (Merck) and then fixed in fresh acetone for 10 minutes at room temperature.

Fixation with Formaldehyde

The cover glass is twice covered with a layer of PBS, and the PBS is then sucked off and discarded. Residues of liquid are allowed to drain off on cellulose. The cover glass is then first covered with a layer of 3.5% formaldehyde (Riedel de Haen) in PBS (the liquid is removed again as in the preceding step) and then fixed for 10 minutes with formaldehyde solution at room temperature.

Fixation with Ethanol

The procedure is the same as the previous fixation, 100% ethanol (Merck) being employed instead of the formaldehyde solution.

### Indirect Immunofluorescence

The fixed slides with cells are stained as described in Example 1. In contrast to staining of the tissue sections, slides with cells are rinsed only briefly with PBS/0.2% Tween in a cuvette during washing, and are then incubated in a further cuvette for 5 minutes in PBS-Tween.

Slides with differently cultured HT29-18N2 cells and primate intestine (as a positive control) are then tested with the anti-goblet cell-positive sera, 288 blood donor sera and a number of samples from patients with confirmed colitis ulcerosa (57 sera) or Crohn's disease (80 sera). Incubation is carried out here by the standard incubation with altered washing steps.

### Results:

The HT29-18N2 cells cultured on cover glasses in DMEgal and fixed with acetone, formaldehyde or ethanol are tested in indirect immunofluorescence for their reactivity with goblet cell-positive sera and sera from blood donors. The use of ethanol and formaldehyde is only poorly suitable for fixing these cells to cover glasses. On all the fields of the slide, the cells become detached during the incubation, so that only a poor conclusion can be made regarding the reactivity of the cells with the sera used. Acetone fixing is therefore carried out for all the following experiments.

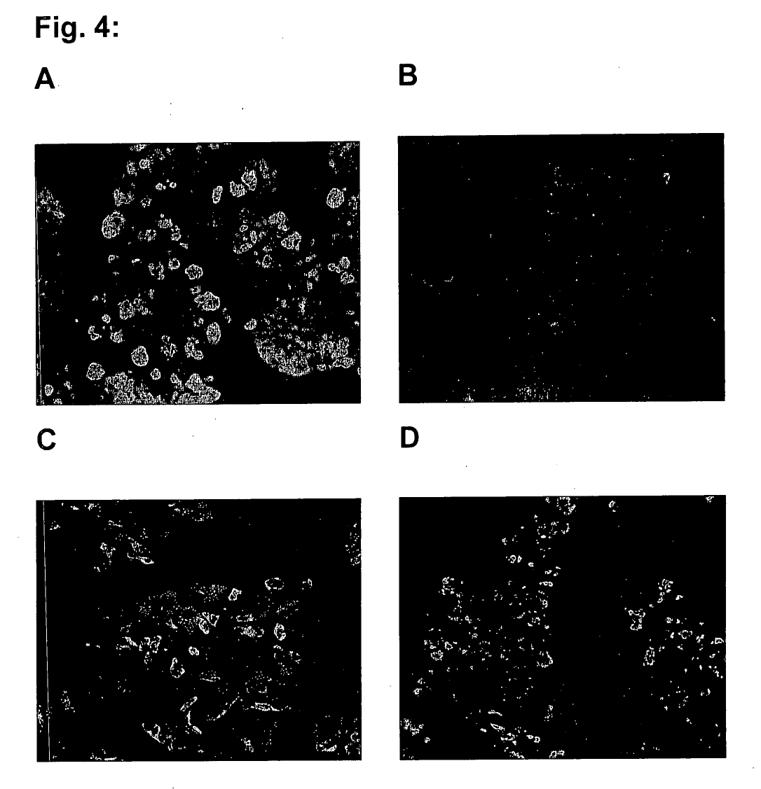

The incubation of cells grown in Protein Free Hybridoma Medium II (PFHM II) and fixed with acetone at various points in time with anti-goblet cell-positive sera shows a cloud-like, blurred fluorescence in a plane above the fixed cells (FIG. 4A). After incubation of the HT29-18N2 cells with blood donor sera, only the cells themselves have a weak, uniform fluorescence (FIG. 4B).

Since only anti-goblet cell-positive sera cause the cloud-like fluorescence pattern similar to the goblet cell-positive reaction of fixed intestinal tissue, the reaction is to be regarded as specific. The longer the cells have grown in the hybridoma medium, the more intense is the specific signal. After growth in PFHM II for 4 days, the cells are particularly suitable for detection of the antigen in indirect immunofluorescence. Cells grown in DMEM/10% FCS or DMEgal and tested in parallel show no specific reactivity.

The 288 sera from blood donors and Crohn's disease patients tested in the indirect immunofluorescence in parallel on primate intestine and HT29-18N2 cells show no goblet cell-specific reactivity. The comparison of the primate intestine and cell line substrates shows that non-specific background fluorescences, which can make the diagnosis by means of intestine difficult for some sera, interferes considerably less with the evaluation of the cells. In addition, in the case of incubation of sera from patients with colitis ulcerosa with positive reactions the cloud-like fluorescence pattern on the cells can be demarcated from the background considerably better.

On comparison of the cells grown in various media, a change in the size and morphology can be seen after changing to PFHM II. The cells appear to increase in size due to a differentiation, and the cytoplasm looks considerably more structured.

### Example 9

### Selectivity of the Binding of Anti-Goblet Cell Antibodies to Goblet Cell Antigen from HT29-18N2 Cells

Further antibodies were tested on the cell line in indirect immunofluorescence. The anti-MUC2 antibody reacts with the cytoplasm of some cells and therefore also shows a fluorescence pattern similar to that on intestinal tissue (FIG. 4C). The anti-MUC3 antibody shows a uniform fluorescence of the cells, regardless of whether they are grown in DMEM or PFHM II, and therefore also reacts with the non-differentiated cells. The reaction of the anti-MUC5AC antibody is limited to the cells grown in PFHM II. Apart from a granular pattern in many cells, a fluorescence manifests itself here in a plane above the cells, which can be distinguished visually from the anti-goblet cell-specific signal (FIG. 4D).

Sera from patients with defined diseases, which react with structures of the HEp-2 cells (human epithelial cells) employed for autoimmune diagnostics, are tested in order to render conclusions regarding a demarcation of different fluorescence patterns possible. These sera contain autoantibodies against cell nuclei (homogeneous, granular and nucleolar ANA pattern), the nucleus membrane, mitochondria (AMA), the Golgi apparatus, vimentin and actin (cytoskeletal structures) and against the antigen Jo-1. These antibodies react with HT29-18N2 cells, but the arising fluorescence patterns can be distinguished unambiguously from the reaction with anti-goblet cell antibodies. These autoantibodies are thus not cross-reactive with goblet cell antigen.

### Example 10

### Sensitivity of the Binding of Anti-Goblet Cell Antibodies to Goblet Cell Antigen from HT29-18N2 Cells

The sera collections from patients with colitis ulcerosa still available in this series of experiments (57 sera) are unsuitable for determining a prevalence of the anti-goblet cell antibody, since mainly positive sera were removed on the basis of early study purposes and the collections can therefore no longer be regarded as representative. Since the sera are pre-characterized with respect to antibodies against goblet cells, and it was possible to determine a prevalence of 28% with these samples with human fetal intestine as the substrate, a comparison can be made between the fetal intestine, the HT29-18N2 cells and the adult primate intestine employed.

### Results:

Of the 57 sera from colitis ulcerosa patients still available, 11 tested positive for antibodies against goblet cells on fetal intestine (Table 3). These 11 sera also reacted specifically to goblet cell on the HT29-18N2 cells. Only 8 sera with antibodies against goblet cells can be determined with the aid of the adult primate intestine. The marked similarity of the fluorescence pattern and a comparison of the sensitivity of the substrates in indirect immunofluorescence therefore show that the HT29-18N2 cell line is more suitable as a substrate for diagnostics for detecting anti-goblet cell antibodies than primate intestine.

TABLE 3
Comparison of the reactivity of HT29-18N2 cells, human fetal
and adult intestine in indirect immunofluorescence after
incubation with patient sera (1:10 in PBS-Tween).

| | | Adult | Fetal |
|---|---|---|---|
| | HT29-18N2 | intestine | intestine |
| positive | 11 | 8 | 11 |
| negative | 46 | 49 | 46 |

### Example 11

### Purification of Goblet Cell Antigen

Since the immunofluorescence shows a reaction in a plane above the cells in the positive case, the supernatant of the cell culture is employed for further tests. The medium from the cell culture flasks of the HT29-18N2 cells in PFHM II is collected, e.g. via a gas wash bottle, concentrated with a 100 kDa cutoff (Vivaspin 100, VivaScience), exchanged for PBS and tested in ELISA or dot blot.

The supernatants obtained from the cell culture are tested for specific reactions in dot blot. For this, twice, each time 1 μl of the cell culture supernatant (after concentration with a 100 kDa cutoff and exchange of the medium for PBS) are directly applied to a previously marked position on the nitrocellulose membrane. In between and thereafter, the membrane is allowed to dry thoroughly. Subsequent incubation with various anti-goblet cell-positive and blood donor sera substantially corresponds to a standard western blot incubation, although the conjugate incubation is prolonged from 30 minutes to one hour. The success of the differentiation is checked in parallel with the anti-MUC5AC antibody, all the cell culture supernatants being positive.

### Results:

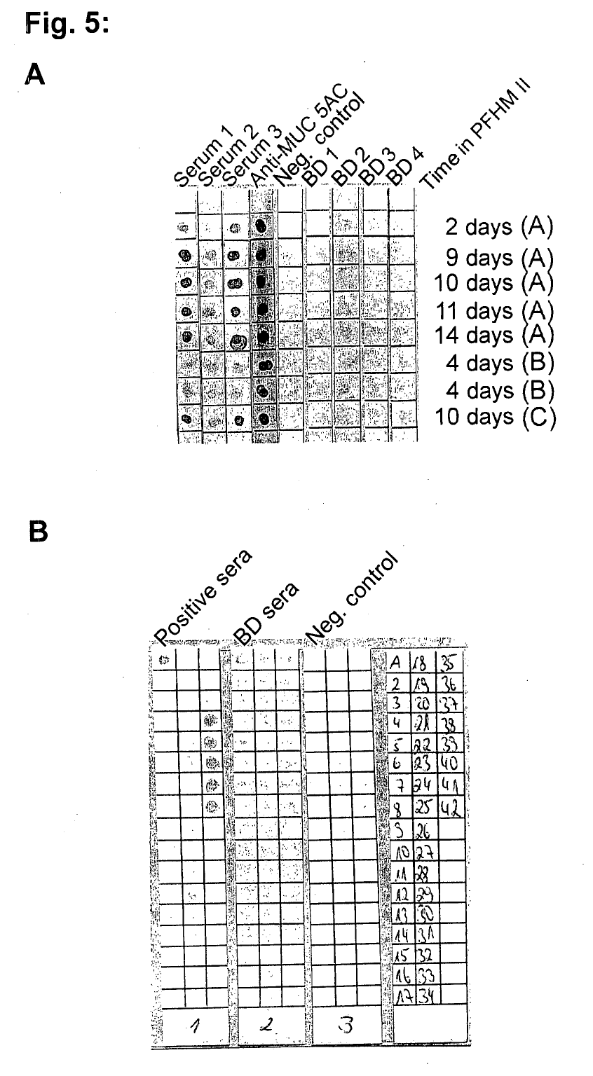

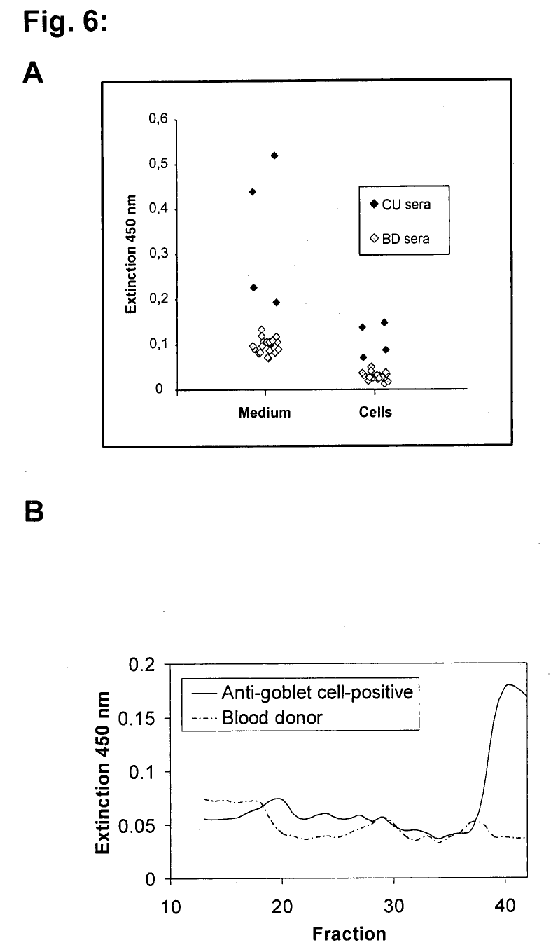

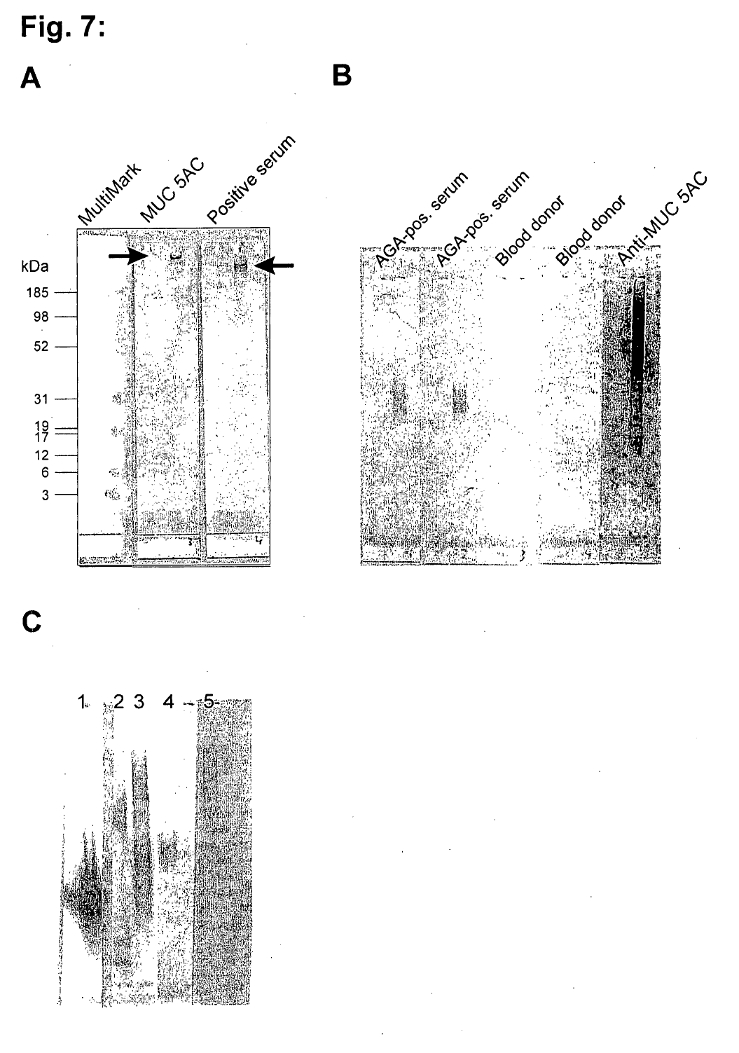

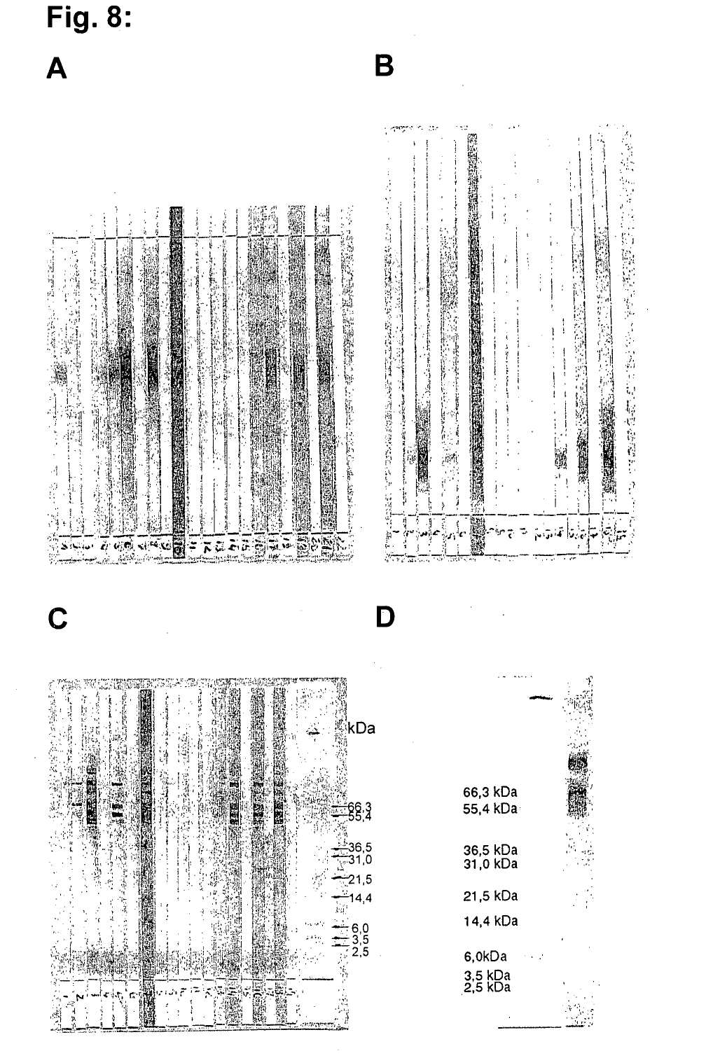

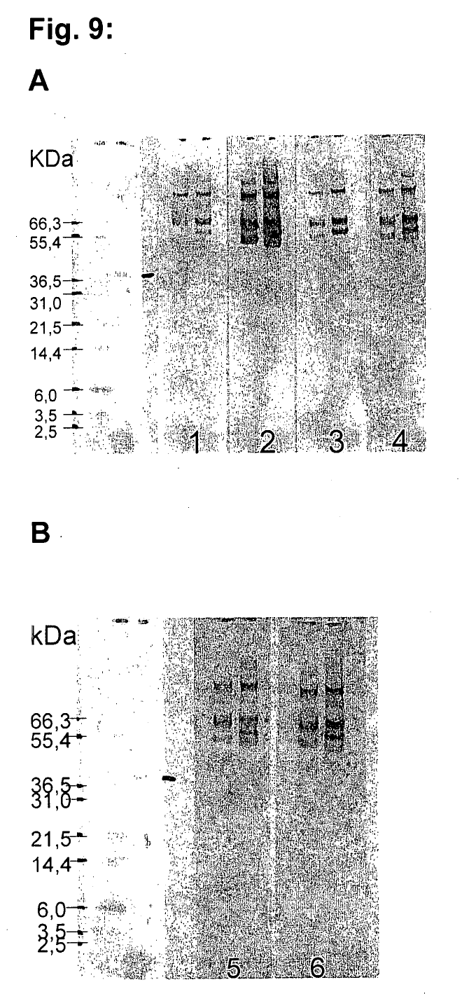

The anti-goblet cell-positive sera show different degrees of reactivity with the individual samples (FIG. 5A). The concentration of the antigen here seems to depend on the time for which the cells have grown in PFHM II medium. 2 days after changing the medium from DMEM to PFHM II, the signal is indeed present, but of weaker intensity. The longer the cells have grown in PFHM II, the more intense is the reaction. The anti-goblet cell-positive sera likewise react to different degrees. The intensity seems to correlate with the titer in the immunofluorescence.

The blood donor sera do not react with any of the cell culture supernatants. The negative control, which was incubated with universal buffer+3% (w/v) of powdered milk instead of a serum dilution likewise shows no reactivity.

### Example 12

### Chromatographic Purification of the Goblet Cell Antigen

The cell culture supernatants from the cell culture were separated on the basis of different charges in the molecules with the aid of anion exchange chromatography. A purification of the protein mixture contained in the samples is thus to be achieved, in order to render the development of a test on the basis of the antigen present in the purest possible form possible (for ELISA, western blotting).

The column used is a POROS HQ/M 20 μm from Applied Biosystems of 16 mm diameter and 10 ml column volume. A BioLogic Duo Flow from Biorad is used as the HPLC unit. The flow rate is 10 ml/min. Fractions of 5 ml volume are collected.

The column is first flushed with 50 ml of water. A regeneration with 50 ml of regeneration buffer (10% acetic acid and 1 M NaCl) then follows. The column is flushed first with 50 ml of water, thereafter with 10 ml of elution buffer (25 mM Tris-HCl pH 8, 250 mM NaCl, 1 mM PMSF) and then with 80 ml of equilibration buffer (25 mM Tris-HCl pH 8, 1 mM PMSF), before application of the sample (5 ml) takes place. The fractions are collected after this point in time. After application of the sample, 50 ml of equilibration buffer are passed over the column. The column is eluted via a gradient which comprises three stages, in each of which the concentration of the chloride ions on the column is increased. In this process, 75, 150 and 250 mM NaCl are employed.

The collected fractions are analyzed for their reactivity with anti-goblet cell-positive and blood donor sera by dot blot in order to be able to make a conclusion regarding the presence of the target antigen.

### Results:

The anti-goblet cell-positive sera react with the sample applied (A) and with the last fractions of the chromatography (FIG. 5B). The blood donor sera react neither with the sample applied nor with one of the fractions. The reaction of the anti-goblet cell-positive sera is thus to be rated as specific. The negative control, which was incubated with universal buffer+3% (w/v) powdered milk instead of a serum dilution, shows no reactivity.

### Example 13

### Detection of Anti-Goblet Cell-Positive Sera in ELISA with Goblet Cell Antigen

By direct coating of an ELISA plate with goblet cell antigen from cell culture supernatant of HT29-18N2 cells and HT29-18N2 cells (in each case after culture in protein-free medium), the reactivity of the fractions is to be tested with a mixture of anti-goblet cell-positive and blood donor sera. The fractions of the chromatography are furthermore also tested in ELISA in addition to dot blot.

The plates are coated overnight at 4° C. or for 2 hours at room temperature with the goblet cell antigen or the cell lysate. The cell lysate is prepared by freezing and thawing the cells in TNE-T buffer. The ELISA is further carried out by standard methods, similar to the lectin ELISA described in Example 6.

### Results:

The preparation with cell lysate only shows a low specific reaction. The cell culture supernatant tested shows an increased reactivity with the anti-goblet cell-positive sera, a correlation being identified between the extinction value and the titer of the corresponding serum in the indirect immunofluorescence. The sera with the highest titers determined by immunofluorescence are also more reactive in the ELISA by comparison, and lead to higher extinction values. Since the blood donor sera show no comparable reaction, the signal is to be evaluated as specific. It is clearly shown in this experiment that the purified goblet cell antigen is more suitable for a detection in ELISA than the cells themselves (FIG. 6A).

The values determined for the fractions of the chromatographic separation with anti-goblet cell-positive sera in dot blot can be confirmed in the ELISA (FIG. 6B).

### Example 14

### Characterization of the Goblet Cell Antigen and Detection in SDS-PAGE/Western Blot

The fractions of the anion exchange chromatography which show a specific reactivity with anti-goblet cell-positive sera in dot blot are separated by means of SDS-PAGE, the gel electrophoresis being carried out under reducing (e.g. after reduction e.g. with mercaptoethanol or dithiothreitol) and non-reducing conditions. MultiMark is used as the marker. Western blotting is carried out, wherein anti-goblet cell-positive sera and, as a control, MUC5AC antibodies are used.

### Results:

Under non-reducing conditions, a band which lies above the top band of the marker (185 kDa) is detected here on incubation with anti-goblet cell-positive sera (FIG. 7A). A band which is above both the top marker band and the band which reacts with the anti-goblet cell-positive serum is also seen after incubation with the anti-MUC5AC antibody. The apparent molecular mass (or the molecular weight) of goblet cell antigen is thus between 185 kDa and the apparent molecular weight of the non-reduced mucin MUC5AC (>2,000 kDa). The results furthermore indicate that the preparation of the goblet cell antigen is contaminated with MUC5AC after the anion exchange chromatography. This experiment also shows that western blotting after electrophoretic separation under non-reducing conditions is a suitable method for detecting anti-goblet cell antibodies in a sample.

Under reducing conditions, neither the goblet cell antigen nor MUC5AC can be detected with the antibodies.

### Example 15

### Characterization of the Goblet Cell Antigen and Detection in Agarose Gel Electrophoresis/Western Blot

Because of the size of the antigen, it is furthermore analyzed in agarose gel electrophoresis. The optimum separation range of the method is approx. 100 to 2,000 kDa.

For the preparation of a gel, 1% (w/v) of agarose and 357 mM bis-Tris-HCl buffer (pH 6.5) are heated briefly until the agarose has dissolved completely.

The mixture is introduced into 10×10 cm empty cassettes (anamed Elektrophorese GmbH), the pocket being shaped with the aid of a comb (anamed Elektrophorese GmbH). After cooling, the agarose gel can be employed analogously to the SDS-PAGE method. The sample preparation is also identical in both cases.

After the separation of proteins in the gel electrophoresis, the proteins, as is known for polyacrylamide gels, can be transferred to a nitrocellulose membrane. The blotting time chosen here can be somewhat shorter, in the wet blotting method used, e.g., only 45 minutes.

### Results:

In the separation of the chromatography fractions by means of agarose gel electrophoresis and subsequent western blotting—as in the SDS-PAGE—no reactivities of the goblet cell antigen with the specific antibody can be detected under reducing conditions (see FIG. 7B). MUC 5AC also can be detected under non-reducing conditions only. Under non-reducing conditions, in each case only one band can be seen after incubation with various anti-goblet cell-positive sera. This is found at the same level for all the sera. After incubation with the anti-MUC5AC antibody, a colored region can be seen above the band which occurs after incubation with anti-goblet cell-positive sera. A band weak in comparison, which occurs only at a very high concentration of the anti-MUC5AC antibody, is also to be made out below the latter position. Blood donor sera show no reactivity.

### Example 16

### Characterization of the Purity of the Goblet Cell Antigen Preparation

The anion exchange chromatography fractions which are positive for goblet cell antigen are labeled with FITC and investigated for their purity in agarose gel electrophoresis under reducing and non-reducing conditions. This is compared to a western blot of non-labeled fractions. For the labeling, 100 μl of 1 M NaHCO₃ solution and 2 mg of FITC (Sigma), dissolved in 40 μl of DMSO (Hybaid), are added to 900 μl of sample. This preparation is incubated overnight at room temperature under exclusion of light on a shaker (Eppendorf). Thereafter, the preparation is concentrated with a 10 kDa cutoff (Vivaspin 10, VivaScience) and washed several times with 1 ml of PBS each time, until the runnings are colorless.

The sample can be employed in western blotting after an SDS gel electrophoresis. The procedure for the western blot incubation is similar to that of standard western blotting incubation, the serum incubation being omitted and anti-FITC antibodies labeled with alkaline phosphatase (Sigma, 1:10,000 in universal buffer) being employed as the conjugate.

### Results:

With the FITC-labeled samples, individual bands can be seen under both reducing and non-reducing conditions—in each case in different positions.

Under non-reducing conditions, in each case a band is seen which is at the same level as the band which occurs after incubation with anti-goblet cell-positive sera and above the band corresponding to IgM. This band, that is to say the goblet cell antigen, appears to represent the main protein content of the preparation. The apparent molecular weight of the goblet cell antigen seems to lie between that of human IgM (about 950 kDa) and that of non-reduced MUC5AC (>2,000 kDa).

In addition, colorations are seen both at the level of the region stained after incubation with the MUC5AC antibody and at the level of the band corresponding to the IgM. This result confirms that the goblet cell antigen preparation is probably contaminated with MUC5AC. IgM or a protein of similar size could furthermore be present in the preparation.

Under reducing conditions, 3 bands are seen after incubation with the anti-FITC antibody. The top band is at the lower end of the region stained with the MUC5AC antibody. Two further bands are seen below the band corresponding to the non-reduced IgM.

### Example 17

### Characterization of Binding of the Goblet Cell Antigen to Lectins

Lectin western blots are carried out after agarose and polyacrylamide gel electrophoresis under non-reducing and reducing conditions.

The staining of western blots with lectins instead of antibodies or sera is carried out analogously, but universal buffer (EUROIMMUN) with 3% (w/v) BSA is employed for blocking. The biotinylated lectins and the corresponding conjugate (ExtrAvidin alkaline phosphatase conjugates, Sigma), are diluted in universal buffer (lectins 1:2,000, ExtrAvidin conjugate 1:5,000). The incubation time is in each case 30 minutes, and for the substrate incubation 3 minutes are sufficient.

### Results:

In the investigation of non-reduced samples, after agarose gel electrophoresis and incubation of the blot with the lectins PHA-L, PHA-E, RCA, Con A, LCA, PSA and AAL (see also Table 1) a band is seen in each case (FIG. 8A) which is at the same level as the band which can be seen after incubation with anti-goblet cell-positive sera. Under the conditions used, the lectins SNA, HHL, MAL I, SBA, DBA, UEA I, SJA, PNA, WGA, GSL I, PLT I, WGA s+b do not show any reaction, like the sera from healthy blood donors.

In the investigation of reduced samples, an intensive band is to be seen with the lectins PHA-L, PHA-E, RCA, Con A, LCA, PSA and AAL, respectively (FIG. 8B), which is at the same level for all the lectins. A small, less intensely colored band is additionally visible with these lectins, apart from PHA-L. The lectins SNA, HHL, MAL I, SBA, DBA, UEA I, SJA, PNA, WGA, GSL I, PLT I, WGA s+b show no reaction.

After separation of the reduced samples in SDS-PAGE, three intensive bands which appear to have a molecular weight of about 56, 66 and 88 kDa can be distinguished with the lectins PHA-L, PHA-E, RCA, Con A, LCA, PSA and AAL (FIG. 8C). On incubation with PHA-E, Con A, LCA, PSA and AAL, two bands lying close to one another are additionally seen in the range of the molecular weights of 52-54 kDa.

Since the proteins present in the goblet cell antigen fraction, apart from the goblet cell antigen itself, are not bound by the lectins under non-reducing conditions, the proteins detected under reducing conditions appear to be constituents of the goblet cell antigen and not constituents of the other proteins.

### Example 17

### Preparation of the Goblet Cell Antigen from an Agarose Gel and Analysis of Binding to the Lectin PHA-E

An agarose gel electrophoresis is carried out as previously with a non-reduced fraction of the goblet cell antigen after anion exchange chromatography. The two regions of this gel lying at the edge underneath the application pocket are separated off by means of a scalpel. Western blotting with anti-goblet cell-positive serum is carried out with these two gel fragments in order to determine the position of the goblet cell antigen in the gel. After the detection has taken place, the region which contains the goblet cell antigen is cut out from the remaining agarose gel, incubated for 10 minutes with NuPAGE sample buffer (Invitrogen) under reducing conditions and laid in a pocket of a NuPAGE gel. After running the gel, lectin blotting is carried out as previously with the lectin PHA-E, which is the most intensely reactive with the goblet cell antigen.

### Result:

After separation of the reduced sample in SDS-PAGE, three intensive bands which appear to have an apparent molecular weight of about 56, 66 and 88 kDa can be distinguished with the lectin PHA-E (FIG. 8D). The additional two bands lying close to one another in the regions of the apparent molecular weights of 52-54 kDa are not seen and therefore do not appear to be a constituent of the goblet cell antigen. The goblet cell antigen thus comprises one or more peptide chains of 56, 66 and 88 kDa.

### Example 18

### Characterization of the Glycan Structure of Goblet Cell Antigen

Sugar residues linked to the protein via an N-glycosidic bond can be split off with an N-glycosidase. For this, 10 μl of 1% (w/v) SDS are added to 80 μl of the sample and incubation is carried out for 15 minutes at 37° C. 7.6 μl of 10% (w/v) Triton X-100 and 4 μl of N-glycosidase F (4 units, Roche AG) are then added. After incubation at 37° C. overnight, the sample can be employed in the gel electrophoresis.

### Result:

After the deglycosylation of the samples with N-glycosidase F, a reactivity with the lectins PHA-L, PHA-E, RCA, LCA, PSA and AAL is furthermore detected (Con A was not investigated) (FIGS. 9A and B). Corresponding to the undigested sample, in each case at least three intensively colored bands are seen which are at the same level for all the lectins, but which are in each case just below the corresponding band positions before digestion. Two correspondingly smaller bands lying close to one another in the region of the apparent molecular weights of 52-54 kDa are not seen. The intensity of the reaction has decreased only little due to the digestion. Both N- and O-glycosidically bonded sugar residues thus appear to be present in the molecule.

1. A goblet cell antigen, characterized in that the antigen is obtainable through expression by the cell line HT29-18N2 which has differentiated to goblet cells in protein-free medium, and is detectable with anti-goblet cell antibodies from colitis ulcerosa patients.

2. The goblet cell antigen as claimed in claim 1, characterized in that the apparent molecular weight, measured in non-reducing SDS-PAGE, is higher than 185 kDa.

3. The goblet cell antigen as claimed in claims 1, characterized in that the apparent molecular weight of the non-reduced antigen in agarose gel electrophoresis is between 950 kDa and 2,000 kDa.

4. The goblet cell antigen as claimed in claim 1, characterized in that it is detectable with at least one of the lectins PHA-L, PHA-E, RCA, Con A, LCA, PSA and AAL.

5. The goblet cell antigen as claimed in claim 1, characterized in that it is detectable with the lectins PHA-L, PHA-E, RCA, Con A, LCA, PSA and AAL.

6. The goblet cell antigen as claimed in claim 1, characterized in that it is not detectable with the lectins SNA, HHL, MAL I, SBA, DBA, UEA I, SJA, PNA, WGA, GSL I, PTL I, WGA s+b.

7. The goblet cell antigen as claimed in claim 1, characterized in that it comprises galactose, N-acetyl-galactosamine and/or fucose.

8. The goblet cell antigen as claimed in claim 1, characterized in that it comprises N- and O-glycosidically bonded glycans.

9. The goblet cell antigen as claimed in claim 1, characterized in that the glycans are partly or completely removed by chemical or enzymatic methods.

10. The goblet cell antigen as claimed in claim 1, characterized in that the reduced antigen comprises one or more glycosylated proteins of the molecular weight 56, 66 or 80 kDa.

11. The goblet cell antigen as claimed in claim 1, characterized in that the antigen is not detectable with antibodies from Crohn's disease patients.

12. The goblet cell antigen as claimed in claim 1, characterized in that the antigen is expressed by a eukaryotic cell line.

13. The goblet cell antigen as claimed in claim 1, characterized in that the antigen is expressed by a human cell line.

14. The goblet cell antigen as claimed in claim 1, characterized in that the antigen is expressed by a human colon carcinoma cell line.

15. The goblet cell antigen as claimed in claim 1, characterized in that the antigen is expressed by a cell line which is derived from the cell line HT29.

16. The goblet cell antigen as claimed in claim 1, characterized in that the antigen is expressed by the cell line HT29-18N2 which has differentiated to goblet cells.

17. The goblet cell antigen as claimed in claim 1, characterized in that the antigen is purified.

18. The goblet cell antigen as claimed in claim 1, characterized in that the antigen is purified from the cell culture supernatant of the cultured cell line.

19. The goblet cell antigen as claimed in claim 1, characterized in that the antigen is purified from the cultured cell line.

20. A monoclonal antibody, characterized in that it recognizes the goblet cell antigen as claimed in claim 1.

21. A kit for diagnosis of inflammatory bowel diseases, characterized in that it comprises the goblet cell antigen as claimed in claim 1 and optionally the monoclonal antibody as claimed in claim 20.

22. A method for the detection of antibodies against goblet cell antigen, in which a biological sample is contacted with the goblet cell antigen as claimed in claim 1 and binding of antibody to the antigen is detected.

23. The method as claimed in claim 22, characterized in that the binding of the antibodies to the antigen is detected with an ELISA, blot, western blot or dot blot.

24. A method for the detection of antibodies against goblet cell antigen, in which a sample is contacted with a cell line which is derived from the HT29 cell line and has differentiated to goblet cells in protein-free medium, and the binding of antibody to the antigen is detected as claimed in claim 1.

25. The method as claimed in claim 24, characterized in that the cell line which has differentiated to goblet cells in protein-free medium is the cell line HT29-18N2.

26. The method as claimed in claim 24, characterized in that the binding of the antibodies to the antigen is detected with indirect or direct immunofluorescence.

27. A method for diagnosis of inflammatory bowel diseases, in which samples from patients are subjected to a method as claimed in claim 22, a colitis ulcerosa being diagnosed by detection of antibodies against goblet cell antigen.

28. The use of goblet cell antigen as claimed in claim 1 for the preparation of a pharmaceutical composition for treatment of colitis ulcerosa.

29. The use of goblet cell antigen as claimed in claim 1 for ex vivo removal of anti-goblet cell antibodies from the blood of colitis ulcerosa patients.

30. The use as claimed in claim 28, characterized in that the antigen is coupled to an inert matrix.

31. The use as claimed in claim 30, characterized in that the inert matrix is silica gel, alginate, cellulose, pectin or carrageen.

32. The use as claimed in claim 28, characterized in that the antigen is coupled to a cytotoxin.

33. The use as claimed in claim 32, characterized in that the cytotoxin is specific for B lymphocytes.

34. A pharmaceutical composition for treatment of colitis ulcerosa, characterized in that it comprises goblet cell antigen as claimed in claim 1.
